(19) United States
(12) Patent Application Publication
(10) Pub. No.: US 2023/0293249 A1
(43) Pub. Date: Sep. 21, 2023

(54) INFORMATION PROCESSING DEVICE, PROGRAM, LEARNING MODEL, AND METHOD FOR GENERATING LEARNING MODEL

(71) Applicant: SONY GROUP CORPORATION, TOKYO (JP)

(72) Inventors: KAZUTO YOKOYAMA, TOKYO (JP); YOHEI KURODA, TOKYO (JP); TETSUHARU FUKUSHIMA, TOKYO (JP); YUKI ITOTANI, TOKYO (JP); KATSUFUMI SUGIMOTO, TOKYO (JP)

(21) Appl. No.: 18/005,915

(22) PCT Filed: Jun. 29, 2021
(86) PCT No.: PCT/JP2021/024436

§ 371 Date: Jan. 18, 2023

(30) Foreign Application Priority Data
Aug. 4, 2020 (JP) 2020-132532

(51) Int. Cl. A61B 34/32; A61B 34/20

(52) U.S. Cl. CPC A61B 34/32; A61B 34/20; A61B 2034/2065

(57) ABSTRACT

There is provided an information processing device (300) including a control unit (324) that controls a medical arm (102) to autonomously operate using a first learning model generated by machine learning a plurality of state information concerning an operation of the medical arm labeled as an operation that should be avoided.

### FIELD

The present disclosure relates to an information processing device, a program, a learning model, and a learning model generation method.

### BACKGROUND

In recent years, in endoscopic surgery, surgery is performed while imaging the abdominal cavity of a patient using an endoscope and displaying a captured image captured by the endoscope on a display. For example, Patent Literature 1 below discloses a technique for associating control of an arm supporting an endoscope with control of electronic zoom of the endoscope.

### CITATION LIST

### Patent Literature

Patent Literature 1: WO 2018/159328 A

### SUMMARY

### Technical Problem

Incidentally, in recent years, in a medical observation system, development for autonomously operating a robot arm device that supports an endoscope has been advanced. For example, a learning device is caused to perform machine learning of surgery content and the like and information concerning motions of a surgeon or scopist corresponding the surgery content and the like and generate a learning model. Control information for autonomously controlling the robot arm device is generated with reference to the learning model obtained in this way, ae control rule, and the like.

However, it is difficult to appropriately label the motions because of characteristics peculiar to the motions. Therefore, since it is difficult to collect a large amount of information concerning the motions, it is difficult to efficiently construct a learning model concerning the motions.

Therefore, the present disclosure proposes an information processing device, a program, a learning model, and a learning model generation method that can collect a large amount of appropriately labeled data for machine learning and efficiently construct a learning model.

### Solution to Problem

According to the present disclosure, an information processing device is provided. The information processing device includes a control unit that performs control of a medical arm to autonomously operate using a first learning model generated by machine learning a plurality of state information concerning an operation of the medical arm. The plurality of state information are labeled as being an operation that should be avoided.

Moreover, according to the present disclosure, a program for causing a computer to execute control of an autonomous operation of a medical arm using a first learning model generated by machine learning a plurality of of state information concerning an operation of the medical arm is provided. The plurality of state information are labeled as an operation that should be avoided.

Moreover, according to the present disclosure, a learning model for causing a computer to function to control a medical arm to autonomously operate to avoid a state output based on the learning model is provided. The learning model includes information concerning a feature value extracted by machine learning a plurality of state information concerning an operation of the medical arm. The plurality of state information are labeled as an operation that should be avoided.

Moreover, according to the present disclosure, a method of generating a learning model for causing a computer to function to control a medical arm to autonomously operate to avoid a state output based on the learning model is provided. The method includes generating the learning model by machine learning a plurality of state information concerning an operation of the medical arm. The plurality of state information are labeled as an operation that the medical arm should avoid.

### BRIEF DESCRIPTION OF DRAWINGS

FIG. 1 is a diagram illustrating an example of a schematic configuration of an endoscopic surgical system to which a technique according to the present disclosure can be applied.

FIG. 2 is a block diagram illustrating an example of functional configurations of a camera head and a CCU (Camera Control Unit) illustrated in FIG. 1.

FIG. 3 is a schematic diagram illustrating a configuration of an oblique view endoscope according to an embodiment of the present disclosure.

FIG. 4 is a diagram illustrating an example of a configuration of a medical observation system 10 according to the embodiment of the present disclosure.

FIG. 5 is an explanatory diagram for explaining an overview of the embodiment of the present disclosure.

FIG. 6 is a block diagram illustrating an example of a configuration of a learning device 200 according to a first embodiment of the present disclosure.

FIG. 7 is a flowchart illustrating an example of a method of generating a learning model for teaching negative cases according to the first embodiment of the present disclosure.

FIG. 8 is an explanatory diagram for explaining an example of a method of generating a learning model for teaching negative cases according to the first embodiment of the present disclosure.

FIG. 9 is a block diagram illustrating an example of a configuration of a control device 300 according to the first embodiment of the present disclosure.

FIG. 10 is a flowchart illustrating an example of a control method according to the first embodiment of the present disclosure.

FIG. 11 is an explanatory diagram for explaining a control method according to the first embodiment of the present disclosure.

FIG. 12 is an explanatory diagram for explaining a method of generating a teacher model according to a second embodiment of the present disclosure.

FIG. 13 is a flowchart illustrating an example of a control method according to the second embodiment of the present disclosure.

FIG. 14 is an explanatory diagram for explaining the control method according to the second embodiment of the present disclosure.

FIG. 15 is an explanatory diagram (part 1) for explaining a control method according to a third embodiment of the present disclosure.

FIG. 16 is an explanatory diagram (part 2) for explaining the control method according to the third embodiment of the present disclosure.

FIG. 17 is a block diagram illustrating an example of a configuration of an evaluation device 400 according to a fourth embodiment of the present disclosure.

FIG. 18 is a flowchart illustrating an example of an evaluation method according to the fourth embodiment of the present disclosure.

FIG. 19 is an explanatory diagram for explaining the evaluation method according to the fourth embodiment of the present disclosure.

FIG. 20 is an explanatory diagram (part 1) for explaining an example of a display screen according to the fourth embodiment of the present disclosure.

FIG. 21 is an explanatory diagram (part 2) for explaining the example of the display screen according to the fourth embodiment of the present disclosure.

FIG. 22 is a hardware configuration diagram illustrating an example of a computer that implements a function of generating a learning model for teaching negative cases according to an embodiment of the present disclosure.

### DESCRIPTION OF EMBODIMENTS

Preferred embodiments of the present disclosure are explained in detail below with reference to the accompanying drawings. Note that, in the present specification and the drawings, components having substantially the same functional configurations are denoted by the same reference numerals and signs, whereby redundant explanation of the components is omitted. In addition, in the present specification and the drawings, a plurality of components having substantially the same or similar functional configurations are sometimes distinguished by attaching different alphabets after the same reference numerals. However, when it is not particularly necessary to distinguish each of the plurality of components having substantially the same or similar functional configurations, only the same reference numerals and signs are attached.

Note that the explanation is made in the following order.
1. Configuration example of an endoscopic surgery system 5000
1.1 Schematic configuration of the endoscopic surgery system 5000
1.2 Detailed configuration example of a support arm device 5027
1.3 Detailed configuration example of a light source device 5043
1.4 Detailed configuration example of a camera head 5005 and a CCU 5039
1.5 Configuration example of an endoscope 5001
2. Configuration example of a medical observation system 10
3. Background leading to creation of embodiments of the present disclosure
4. First Embodiment
4.1 Generation of a learning model for teaching negative cases
4.2 Autonomous control by the learning model for teaching negative cases
5. Second Embodiment
5.1 Generation of a learning model for teaching negative cases
5.2 Autonomous control by a learning model for teaching negative cases
6. Third Embodiment
7. Fourth Embodiment
7.1 Detailed configuration example of an evaluation device 400
7.2 Evaluation method
8. Summary
9. Hardware configuration
10. Supplement

### 1. Configuration Example of an Endoscopic Surgery System 5000

<1.1 Schematic Configuration of the Endoscopic Surgery System 5000>

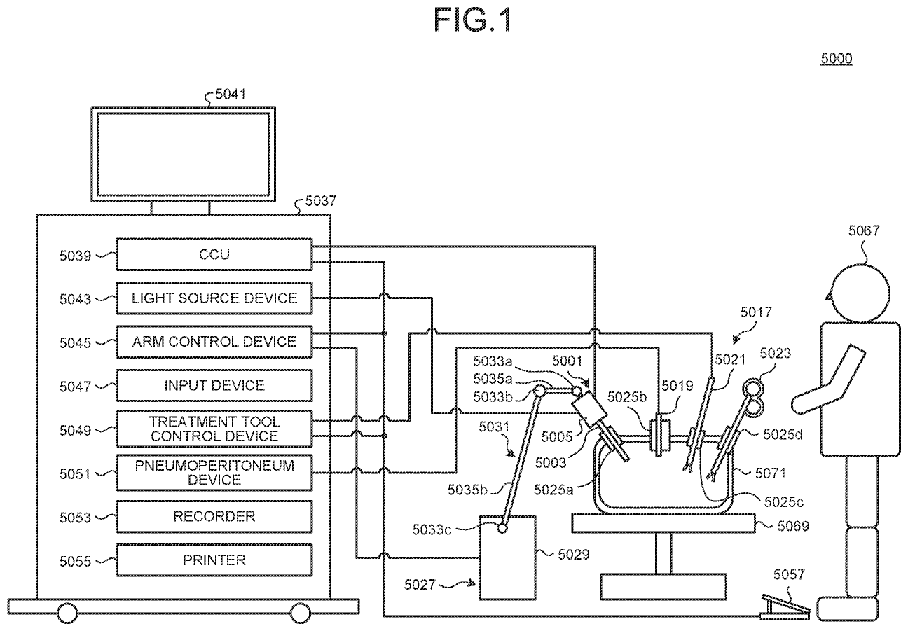

First, before details of embodiments of the present disclosure are explained, a schematic configuration of an endoscopic surgery system 5000 to which the technique according to the present disclosure can be applied is explained with reference to FIG. 1. FIG. 1 is a diagram illustrating an example of a schematic configuration of the endoscopic surgery system 5000 to which the technique according to the present disclosure can be applied. FIG. 1 illustrates a state in which a surgeon 5067 is performing surgery on a patient 5071 on a patient bed 5069 using the endoscopic surgery system 5000. As illustrated in FIG. 1, the endoscopic surgery system 5000 includes an endoscope 5001, other surgical tools (medical instruments) 5017, a support arm device (a medical arm) 5027 that supports the endoscope (a medical observation device) 5001, and a cart 5037 on which various devices for endoscopic surgery are mounted. Details of the endoscopic surgery system 5000 are sequentially explained below.

(Surgical Tools 5017)

In endoscopic surgery, instead of cutting the abdominal wall and opening the abdomen, for example, a plurality of cylindrical puncture instruments called trocars 5025a to 5025d are punctured into the abdominal wall. Then, a lens barrel 5003 of the endoscope 5001 and the other surgical tools 5017 are inserted into the body cavity of the patient 5071 from the trocars 5025a to 5025d. In the example illustrated in FIG. 1, as the other surgical tools 5017, a pneumoperitoneum tube 5019, an energy treatment tool 5021, and forceps 5023 are inserted into the body cavity of the patient 5071. The energy treatment tool 5021 is a treatment tool that performs incision and detachment of a tissue, sealing of a blood vessel, or the like with a high-frequency current or an ultrasonic vibration. However, the surgical tool 5017 illustrated in FIG. 1 is merely an example. Examples of the surgical tool 5017 include various surgical tools generally used in endoscopic surgery such as tweezers and a retractor.

(Support Arm Device 5027)

The support arm device 5027 includes an arm unit 5031 extending from a base portion 5029. In the example illustrated in FIG. 1, the arm unit 5031 includes joint portions 5033a, 5033b, and 5033c and links 5035a and 5035b and is driven by control from an arm control device 5045. Then, the endoscope 5001 is supported by the arm unit 5031 and the position and the posture of the endoscope 5001 are controlled. As a result, stable fixation of the position of the endoscope 5001 can be realized.

(Endoscope 5001)

The endoscope 5001 includes a lens barrel 5003, whose region of a predetermined length from the distal end is inserted into the body cavity of the patient 5071, and a camera head 5005 connected to the proximal end of the lens barrel 5003. In the example illustrated in FIG. 1, the endoscope 5001 configured as a so-called rigid endoscope including a rigid lens barrel 5003 is illustrated. However, the endoscope 5001 may be configured as a so-called flexible endoscope including a flexible lens barrel 5003. In the embodiment of the present disclosure, the endoscope 5001 is not particularly limited.

An opening portion into which an objective lens is fitted is provided at the distal end of the lens barrel 5003. A light source device 5043 is connected to the endoscope 5001. Light generated by the light source device 5043 is guided to the distal end of the lens barrel by a light guide extended to the inside of the lens barrel 5003 and is irradiated toward an observation target in the body cavity of the patient 5071 via the objective lens. Note that, in the embodiment of the present disclosure, the endoscope 5001 may be a front direct view endoscope or an oblique view endoscope and is not particularly limited.

An optical system and an imaging element are provided inside the camera head 5005. Reflected light (observation light) from an observation target is condensed on the imaging element by the optical system. The observation light is photoelectrically converted by the imaging element. An electric signal corresponding to the observation light, that is, an image signal corresponding to an observation image is generated. The image signal is transmitted to a camera control unit (CCU) 5039 as RAW data. Note that the camera head 5005 is implemented with a function of adjusting magnification and a focal length by appropriately driving the optical system.

Note that, for example, in order to cope with a stereoscopic view (3D display) or the like, a plurality of imaging elements may be provided in the camera head 5005. In this case, a plurality of relay optical systems are provided inside the lens barrel 5003 in order to guide the observation light to each of the plurality of imaging elements.

(Various Devices Mounted on a Cart)

First, a display device 5041 displays, according to control of the CCU 5039, an image based on an image signal subjected to image processing by the CCU 5039. When the endoscope 5001 is adapted to high-resolution imaging such as 4K (the number of horizontal pixels 3840×the number of vertical pixels 2160) or 8K (the number of horizontal pixels 7680×the number of vertical pixels 4320) and/or when the endoscope 5001 is adapted to 3D display, for example, a display device capable of performing high-resolution display and/or a display device capable of performing 3D display corresponding the endoscope 5001 is used as the display device 5041. A plurality of display devices 5041 having different resolutions and sizes may be provided according to uses.

An image of a surgical site in the body cavity of the patient 5071 captured by the endoscope 5001 is displayed on the display device 5041. While viewing, in real time, the image of the surgical site displayed on the display device 5041, the surgeon 5067 can perform treatment for, for example, resecting of an affected part using the energy treatment tool 5021 and the forceps 5023. Note that, although not illustrated, the pneumoperitoneum tube 5019, the energy treatment tool 5021, and the forceps 5023 may be supported by the surgeon 5067, an assistant, or the like during surgery.

The CCU 5039 includes a CPU (Central Processing Unit), a graphics processing unit (GPU), and the like and can collectively control the operation of the endoscope 5001 and the display device 5041. Specifically, the CCU 5039 performs, on an image signal received from the camera head 5005, various kinds of image processing for displaying an image based on the image signal such as development processing (demosaic processing). Further, the CCU 5039 provides the image signal subjected to the image processing to the display device 5041. The CCU 5039 transmits a control signal to the camera head 5005 and controls driving of the camera head 5005. The control signal can include information concerning imaging conditions such as magnification and a focal length.

The light source device 5043 includes a light source such as an LED (Light Emitting Diode) and supplies irradiation light in photographing a surgical site to the endoscope 5001.

The arm control device 5045 includes a processor such as a CPU and operates according to a predetermined program to thereby control driving of the arm unit 5031 of the support arm device 5027 according to a predetermined control scheme.

An input device 5047 is an input interface to the endoscopic surgery system 5000. The surgeon 5067 can input various kinds of information and instructions to the endoscopic surgery system 5000 via the input device 5047. For example, the surgeon 5067 inputs various types of information concerning surgery such as physical information of a patient and information concerning a surgical procedure of surgery via the input device 5047. For example, the surgeon 5067 can input an instruction to drive the arm unit 5031, an instruction to change imaging conditions (a type, magnification, a focal length, and the like of irradiation light) by the endoscope 5001, an instruction to drive the energy treatment tool 5021, and the like via the input device 5047. Note that a type of the input device 5047 is not limited. The input device 5047 may be various publicly-known input devices. As the input device 5047, for example, a mouse, a keyboard, a touch panel, a switch, a foot switch 5057, a lever, and/or the like can be applied. For example, when a touch panel is used as the input device 5047, the touch panel may be provided on a display surface of the display device 5041.

Alternatively, the input device 5047 may be a device worn on a part of the body of the surgeon 5067 such as a glasses-type wearable device or an HMD (Head Mounted Display). In this case, various inputs are performed according to a gesture or a line of sight of the surgeon 5067 detected by these devices. The input device 5047 can include a camera capable of detecting a movement of the surgeon 5067. Various inputs may be performed according to a gesture or a line of sight of the surgeon 5067 detected from an image captured by the camera. Further, the input device 5047 can include a microphone capable of collecting voice of the surgeon 5067. Various inputs may be performed by voice via the microphone. As explained above, the input device 5047 is configured to be capable of inputting various kinds of information in a non-contact manner. Therefore, in particular, a user (for example, the surgeon 5067) belonging to a clean area can operate equipment belonging to an unclean area in a non-contact manner. Since the surgeon 5067 can operate the equipment without releasing his/her hand from a held surgical tool, the convenience of the surgeon 5067 is improved.

A treatment tool control device 5049 controls driving of the energy treatment tool 5021 for cauterization and incision of a tissue, sealing of a blood vessel, or the like. A pneumoperitoneum device 5051 feeds gas into the body cavity of the patient 5071 via the pneumoperitoneum tube 5019 in order to inflate the body cavity for the purpose of securing a visual field by the endoscope 5001 and securing a working space of the surgeon 5067. A recorder 5053 is a device capable of recording various kinds of information concerning surgery. A printer 5055 is a device capable of printing various kinds of information concerning surgery in various formats such as text, image, or graph.

<1.2 Detailed Configuration Example of the Support Arm Device 5027>

Further, an example of a detailed configuration of the support arm device 5027 is explained. The support arm device 5027 includes a base portion 5029, which is a base, and an arm unit 5031 extending from the base portion 5029. In the example illustrated in FIG. 1, the arm unit 5031 includes a plurality of joint portions 5033a, 5033b, and 5033c and a plurality of links 5035a and 5035b coupled by the joint portion 5033b. However, in FIG. 1, the configuration of the arm unit 5031 is illustrated in a simplified manner for the sake of simplicity. Specifically, the shape, the number, and the disposition of the joint portions 5033a to 5033c and the links 5035a and 5035b, the direction of the rotation axes of the joint portions 5033a to 5033c, and the like can be set as appropriate such that the arm unit 5031 has a desired degree of freedom. For example, the arm unit 5031 can be suitably configured to have 6 degrees of freedom or more. Consequently, since the endoscope 5001 can be freely moved within a movable range of the arm unit 5031, the lens barrel 5003 of the endoscope 5001 can be inserted into the body cavity of the patient 5071 from a desired direction.

Actuators are provided in the joint portions 5033a to 5033c. The joint portions 5033a to 5033c are configured to be rotatable around a predetermined rotation axis according to driving of the actuators. The driving of the actuators is controlled by the arm control device 5045, whereby rotation angles of the joint portions 5033a to 5033c is controlled and the driving of the arm unit 5031 is controlled. Consequently, control of the position and the posture of the endoscope 5001 can be realized. At this time, the arm control device 5045 can control the driving of the arm unit 5031 with various publicly-known control methods such as force control or position control.

For example, the surgeon 5067 performs operation input as appropriate via the input device 5047 (including the foot switch 5057), whereby the driving of the arm unit 5031 may be controlled as appropriate by the arm control device 5045 according to the operation input and the position and the posture of the endoscope 5001 may be controlled. Note that the arm unit 5031 may be operated in a so-called master-slave scheme. In this case, the arm unit 5031 (a slave) can be remotely controlled by the surgeon 5067 via the input device 5047 (a master console) set in a place away from the operating room or in the operating room.

Here, in general, in endoscopic surgery, the endoscope 5001 is supported by a doctor called scopist. In contrast, in the embodiment of the present disclosure, since the position of the endoscope 5001 can be more reliably fixed without manual operation by using the support arm device 5027, an image of a surgical site can be stably obtained and surgery can be smoothly performed.

Note that the arm control device 5045 may not necessarily be provided in the cart 5037. The arm control device 5045 may not necessarily be one device. For example, the arm control device 5045 may be provided in each of the joint portions 5033a to 5033c of the arm unit 5031 of the support arm device 5027. Drive control of the arm unit 5031 may be realized by a plurality of arm control devices 5045 cooperating with each other.

<1.3 Detailed configuration example of the light source device 5043>

Subsequently, an example of a detailed configuration of the light source device 5043 is explained. The light source device 5043 supplies irradiation light in photographing a surgical site to the endoscope 5001. The light source device 5043 includes, for example, an LED, a laser light source, or a white light source including a combination the LED and the laser light source. At this time, when the white light source is configured by a combination of RGB laser light sources, output intensities and output timings of colors (wavelengths) can be controlled with high accuracy. Therefore, a white balance of a captured image can be adjusted in the light source device 5043. In this case, by irradiating an observation target with laser light from each of the RGB laser light sources in a time division manner and controlling the driving of the imaging element of the camera head 5005 in synchronization with the irradiation timing, it is also possible to capture an image corresponding to each of RGB in a time division manner. According to the method, a color image can be obtained even if a color filter is not provided in the imaging element.

Driving of the light source device 5043 may be controlled to change the intensity of output light at every predetermined time. By controlling the driving of the imaging element of the camera head 5005 in synchronization with the timing of the change of the light intensity to acquire images in a time division manner and combining the images, it is possible to generate an image in a high dynamic range without so-called black solid and white void.

Furthermore, the light source device 5043 may be configured to be capable of supplying light in a predetermined wavelength band adapted to special light observation. In the special light observation, for example, so-called narrow band imaging for imaging a predetermined tissue such as a blood vessel in a mucosal surface layer with high contrast by irradiating light in a narrower band compared with irradiation light (that is, white light) at the time of normal observation using wavelength dependency of light absorption in a body tissue is performed. Alternatively, in the special light observation, fluorescence observation for obtaining an image with fluorescent light generated by irradiation with excitation light may be performed. In the fluorescence observation, for example, fluorescence observation for irradiating a body tissue with excitation light and observing fluorescent light from the body tissue (autofluorescence observation) or fluorescence observation for locally injecting a reagent such as indocyanine green (ICG) into a body tissue and irradiating the body tissue with excitation light corresponding to a fluorescence wavelength of the reagent to obtain a fluorescent image can be performed. The light source device 5043 can be configured to be capable of supplying narrow band light and/or excitation light corresponding to such special light observation.

<1.4 Detailed Configuration Example of the Camera Head 5005 and the CCU 5039>

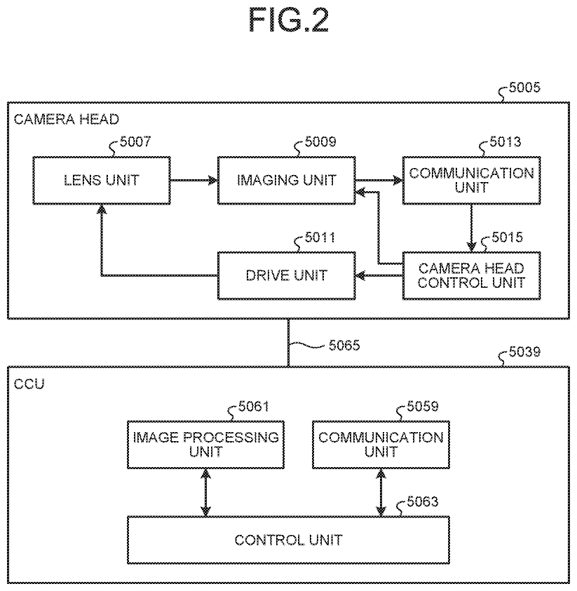

Subsequently, an example of detailed configurations of the camera head 5005 and the CCU 5039 is explained with reference to FIG. 2. FIG. 2 is a block diagram illustrating an example of functional configurations of the camera head 5005 and the CCU 5039 illustrated in FIG. 1.

Specifically, as illustrated in FIG. 2, the camera head 5005 includes, as functions thereof, a lens unit 5007, an imaging unit 5009, a drive unit 5011, a communication unit 5013, and a camera head control unit 5015. The CCU 5039 includes a communication unit 5059, an image processing unit 5061, and a control unit 5063 as functions thereof. The camera head 5005 and the CCU 5039 are connected to be bidirectionally communicable by a transmission cable 5065.

First, a functional configuration of the camera head 5005 is explained. The lens unit 5007 is an optical system provided in a connection portion to the lens barrel 5003. Observation light taken in from the distal end of the lens barrel 5003 is guided to the camera head 5005 and is made incident on the lens unit 5007. The lens unit 5007 is configured by combining a plurality of lenses including a zoom lens and a focus lens. The optical characteristics of the lens unit 5007 are adjusted to condense the observation light on a light receiving surface of the imaging element of the imaging unit 5009. The zoom lens and the focus lens are configured such that positions on optical axes thereof are movable in order to adjust the magnification and the focal point of a captured image.

The imaging unit 5009 includes an imaging element and is disposed at a post stage of the lens unit 5007. The observation light having passed through the lens unit 5007 is condensed on the light receiving surface of the imaging element and an image signal corresponding to an observation image is generated by photoelectric conversion. The image signal generated by the imaging unit 5009 is provided to the communication unit 5013.

As the imaging element configuring the imaging unit 5009, for example, a CMOS (Complementary Metal Oxide Semiconductor) type image sensor having a Bayer array and capable of performing color photographing is used. Note that, as the imaging element, for example, an imaging element adaptable to photographing of an image with high resolution of 4K or more may be used. Since an image of a surgical site is obtained with high resolution, the surgeon 5067 can grasp a state of the surgical site in more detail and can proceed with surgery more smoothly.

The imaging element configuring the imaging unit 5009 may be configured to include a pair of imaging elements for respectively ACQUIRING right-eye and left-eye image signals corresponding to 3D display (a stereo scheme). Since the 3D display is performed, the surgeon 5067 is capable of more accurately grasping the depth of a biological tissue (organ) in the surgical site and grasp the distance to the biological tissue. Note that, when the imaging unit 5009 is configured as a multi-plate type, a plurality of systems of lens units 5007 may be provided to correspond to the imaging elements.

The imaging unit 5009 may not be necessarily provided in the camera head 5005. For example, the imaging unit 5009 may be provided immediately behind the objective lens inside the lens barrel 5003.

The drive unit 5011 includes an actuator and moves the zoom lens and the focus lens of the lens unit 5007 by a predetermined distance along the optical axis according to the control of the camera head control unit 5015. consequently, the magnification and the focus of a captured image captured by the imaging unit 5009 can be adjusted as appropriate.

The communication unit 5013 includes a communication device for transmitting and receiving various kinds of information to and from the CCU 5039. The communication unit 5013 transmits an image signal obtained from the imaging unit 5009 to the CCU 5039 via the transmission cable 5065 as RAW data. At this time, in order to display a captured image of a surgical site with low latency, the image signal is preferably transmitted by optical communication. This is because, at the time of surgery, since the surgeon 5067 performs surgery while observing a state of an affected part with a captured image, for safer and more reliable surgery, it is required to display a moving image of the surgical site in real time as much as possible. When optical communication is performed, a photoelectric conversion module that converts an electric signal into an optical signal is provided in the communication unit 5013. The image signal is converted into an optical signal by the photoelectric conversion module and then transmitted to the CCU 5039 via the transmission cable 5065.

The communication unit 5013 receives, from the CCU 5039, a control signal for controlling driving of the camera head 5005. The control signal includes information concerning imaging conditions such as information indicating that a frame rate of a captured image is designated, information indicating that an exposure value at the time of imaging is designated, and/or information indicating that magnification and a focus of the captured image is designated. The communication unit 5013 provides the received control signal to the camera head control unit 5015. Note that a control signal from the CCU 5039 may also be transmitted by optical communication. In this case, a photoelectric conversion module that converts an optical signal into an electric signal is provided in the communication unit 5013. The control signal is converted into an electric signal by the photoelectric conversion module and then provided to the camera head control unit 5015.

Note that the imaging conditions such as the frame rate, the exposure value, the magnification, and the focus are automatically set by the control unit 5063 of the CCU 5039 based on the acquired image signal. That is, a so-called AE (Auto Exposure) function, a so-called AF (Auto Focus) function, and a so-called AWB (Auto White Balance) function are implemented in the endoscope 5001.

The camera head control unit 5015 controls driving of the camera head 5005 based on the control signal from the CCU 5039 received via the communication unit 5013. For example, the camera head control unit 5015 controls driving of the imaging element of the imaging unit 5009 based on the information indicating that the frame rate of the captured image is designated and/or the information indicating that the exposure at the time of imaging is designated. For example, the camera head control unit 5015 moves the zoom lens and the focus lens of the lens unit 5007 via the drive unit 5011 as appropriate based on the information indicating that the magnification and the focal point of the captured image are designated. The camera head control unit 5015 may further include a function of storing information for identifying the lens barrel 5003 and the camera head 5005.

Note that, by disposing the components such as the lens unit 5007 and the imaging unit 5009 in a sealed structure having high airtightness and waterproofness, the camera head 5005 can have resistance to autoclave sterilization treatment.

Subsequently, a functional configuration of the CCU 5039 is explained. The communication unit 5059 includes a communication device for transmitting and receiving various kinds of information to and from the camera head 5005. The communication unit 5059 receives an image signal transmitted from the camera head 5005 via the transmission cable 5065. At this time, as explained above, the image signal can be suitably transmitted by optical communication. In this case, a photoelectric conversion module that converts an optical signal into an electrical signal is provided in the communication unit 5059 to be adapted to the optical communication. The communication unit 5059 provides the image signal converted into the electric signal to the image processing unit 5061.

Furthermore, the communication unit 5059 transmits a control signal for controlling driving of the camera head 5005 to the camera head 5005. The control signal may also be transmitted by the optical communication.

The image processing unit 5061 applies various kinds of image processing to the image signal, which is RAW data, transmitted from the camera head 5005. Examples of the image processing include various kinds of publicly-known signal processing such as development processing, high image quality processing (band emphasis processing, super-resolution processing, NR (Noise Reduction) processing, camera shake correction processing, and/or the like), and/or enlargement processing (electronic zoom processing). The image processing unit 5061 performs detection processing on an image signal for performing AE, AF, and AWB.

The image processing unit 5061 includes a processor such as a CPU or a GPU. The processor operates according to a predetermined program, whereby the image processing and the detection processing explained above can be performed. Note that, when the image processing unit 5061 includes a plurality of GPUs, the image processing unit 5061 divides information related to an image signal as appropriate and performs image processing in parallel with the plurality of GPUs.

The control unit 5063 performs various kinds of control concerning imaging of a surgical site by the endoscope 5001 and display of a captured image of the surgical site. For example, the control unit 5063 generates a control signal for controlling driving of the camera head 5005. At this time, when imaging conditions are input by the surgeon 5067, the control unit 5063 generates a control signal based on the input by the surgeon 5067. Alternatively, when the AE function, the AF function, and the AWB function are implemented in the endoscope 5001, the control unit 5063 calculates an optimum exposure value, an optimum focal length, and an optimum white balance according to a result of the detection processing by the image processing unit 5061 and generates a control signal.

The control unit 5063 causes the display device 5041 to display the image of the surgical site based on the image signal subjected to the image processing by the image processing unit 5061. At this time, the control unit 5063 recognizes various objects in the surgical site image using various image recognition techniques. For example, the control unit 5063 can recognize a surgical tool such as forceps, a specific biological site, bleeding, mist at the time of use of the energy treatment tool 5021, and the like by detecting shapes, colors, and the like of edges of the objects included in the surgical site image. When displaying the image of the surgical site on the display device 5041, the control unit 5063 superimposes and displays various kinds of surgery support information on the image of the surgical site using a result of the recognition. The surgery support information is superimposed and displayed and is presented to the surgeon 5067, whereby it is possible to more safely and reliably proceed with the surgery.

The transmission cable 5065 connecting the camera head 5005 and the CCU 5039 is an electric signal cable adapted to electric signal communication, an optical fiber adapted to optical communication, or a composite cable of these cables.

Here, in the illustrated example, wired communication is performed using the transmission cable 5065. However, communication between the camera head 5005 and the CCU 5039 may be performed by radio. When the communication between the camera head 5005 and the CCU 5039 is performed by radio, it is unnecessary to lay the transmission cable 5065 in the operating room. Therefore, a situation in which movement of medical staff (for example, the surgeon 5067) in the operating room is hindered by the transmission cable 5065 can be eliminated.

<1.5 Configuration Example of the Endoscope 5001>

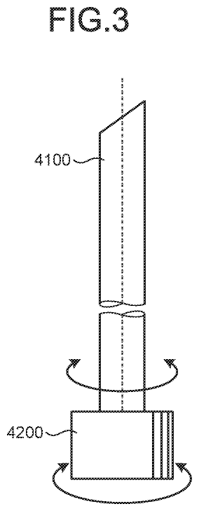

Subsequently, a basic configuration of an oblique view endoscope is explained as an example of the endoscope 5001 with reference to FIG. 3. FIG. 3 is a schematic diagram illustrating a configuration of an oblique view endoscope 4100 according to an embodiment of the present disclosure.

Specifically, as illustrated in FIG. 3, the oblique view endoscope 4100 is attached to the distal end of the camera head 4200. The oblique view endoscope 4100 corresponds to the lens barrel 5003 explained with reference to FIG. 1 and FIG. 2. The camera head 4200 corresponds to the camera head 5005 explained with reference to FIG. 1 and FIG. 2. The oblique view endoscope 4100 and the camera head 4200 are made rotatable independently of each other. An actuator is provided between the oblique view endoscope 4100 and the camera head 4200 in the same manner as among the joint portions 5033a, 5033b, and 5033c. The oblique view endoscope 4100 rotates with respect to the camera head 4200 according to driving of the actuator.

The oblique view endoscope 4100 is supported by the support arm device 5027. The support arm device 5027 has a function of holding the oblique view endoscope 4100 on behalf of the scopist and moving the oblique view endoscope 4100 according to operation of the surgeon 5067 or the assistant such that a desired site can be observed.

Note that, in the embodiment of the present disclosure, the endoscope 5001 is not limited to the oblique view endoscope 4100. For example, the endoscope 5001 may be a front direct view endoscope (not illustrated) that images the front of the distal end portion of the endoscope and may further have a function of cutting out an image from a wide-angle image captured by the endoscope (wide-angle/cutout function). For example, the endoscope 5001 may be an endoscope with a distal end bending function (not illustrated) capable of changing a field of view by freely bending the distal end portion of the endoscope according to operation of the surgeon 5067. For example, the endoscope 5001 may be an endoscope with a simultaneous photographing function in another direction (not illustrated) in which a plurality of camera units having different visual fields are built at the distal end portion of the endoscope and different images can be obtained by the respective cameras.

The example of the endoscopic surgery system 5000 to which the technique according to the present disclosure can be applied is explained above. Note that, here, as an example, the endoscopic surgery system 5000 is explained. However, a system to which the technique according to the present disclosure can be applied is not limited to such an example. For example, the technique according to the present disclosure may be applied to a microscopic surgery system.

### <<2. Configuration Example of the Medical Observation System 10

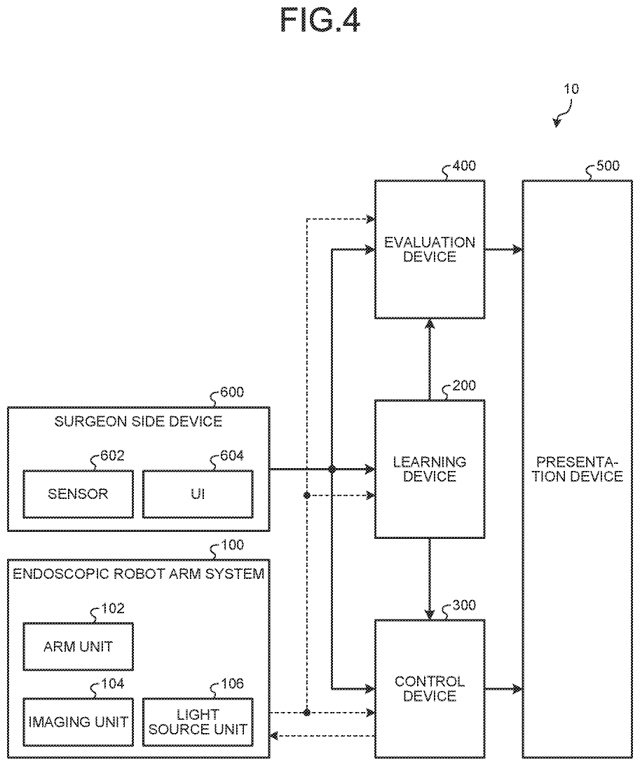

Further, an example of a configuration of the medical observation system 10 according to the embodiment of the present disclosure that can be combined with the endoscopic surgery system 5000 explained above is explained with reference to FIG. 4. FIG. 4 is a diagram illustrating an example of a configuration of the medical observation system 10 according to the embodiment of the present disclosure. As illustrated in FIG. 4, the medical observation system 10 mainly includes an endoscopic robot arm system 100, a learning device 200, a control device 300, an evaluation device 400, a presentation device 500, and a surgeon side device 600. The devices included in the medical observation system 10 are explained below.

First, before details of the configuration of the medical observation system 10 are explained, an overview of an operation of the medical observation system 10 is explained. In the medical observation system 10, by controlling an arm unit 102 (corresponding to the support arm device 5027 explained above) using the endoscopic robot arm system 100, the position of an imaging unit 104 (corresponding to the endoscope 5001 explained above) supported by the arm unit 102 can be fixed at a suitable position without manual operation. Therefore, according to the medical observation system 10, since an image of a surgical site can be stably obtained, the surgeon 5067 can smoothly perform surgery. Note that, in the following explanation, a person who moves or fixes the position of the endoscope is referred to as scopist and an operation of the endoscope 5001 (including movement, stop, change in posture, zoom-in, zoom-out, and the like.) is referred to as scope work irrespective of manual or mechanical control.

(Endoscopic Robot Arm System 100)

The endoscopic robot arm system 100 is the arm unit 102 (the support arm device 5027) that supports the imaging unit 104 (the endoscope 5001) and, specifically, as illustrated in FIG. 4, mainly includes the arm unit (a medical arm) 102, the imaging unit (a medical observation device) 104, and a light source unit 106. The functional units included in the endoscopic robot arm system 100 are explained below.

The arm unit 102 includes an articulated arm (corresponding to the arm unit 5031 illustrated in FIG. 1), which is a multilink structure including a plurality of joint portions and a plurality of links. By driving the arm unit 102 within a movable range, it is possible to control the position and the posture of the imaging unit 104 (the endoscope 5001) provided at the distal end of the arm unit 102. Furthermore, the arm unit 102 may include a motion sensor (not illustrated) including an acceleration sensor, a gyro sensor, and a geomagnetic sensor in order to obtain data of the position and the posture of the arm unit 102.

The imaging unit 104 is provided at the distal end of the arm unit 102 and captures images of various imaging target objects. In other words, the arm unit 102 supports the imaging unit 104. Note that, as explained above, the imaging unit 104 may be, for example, the oblique view endoscope 4100, the front direct view endoscope with the wide angle/cut-out function (not illustrated), the endoscope with the distal end bending function (not illustrated), or the endoscope with the simultaneous imaging function in another direction (not illustrated), or may be a microscope, and is not particularly limited.

Further, the imaging unit 104 can capture, for example, an operative field image including various medical instruments (surgical tools), organs, and the like in the abdominal cavity of a patient. Specifically, the imaging unit 104 is a camera that can photograph a photographing target in forms of a moving image and a still image and is preferably a wide-angle camera including a wide-angle optical system. For example, while an angle of view of a normal endoscope is approximately 80°, an angle of view of the imaging unit 104 according to the present embodiment may be 140°. Note that the angle of view of the imaging unit 104 may be smaller than 140° or may be equal to or larger than 140° if the angle of view exceeds 80°. The imaging unit 104 can transmit an electric signal (an image signal) corresponding to the captured image to the control device 300 or the like. Note that, in FIG. 4, the imaging unit 104 does not need to be included in the endoscopic robot arm system 100. A form of the imaging unit 104 is not limited if the imaging unit 104 is supported by the arm unit 102. Further, the arm unit 102 may support a medical instrument such as the forceps 5023.

In the embodiment of the present disclosure, the imaging unit 104 may be a stereoscopic endoscope capable of performing distance measurement. Alternatively, a depth sensor (a distance measuring device) (not illustrated) may be provided in the imaging unit 104 or separately from the imaging unit 104. The depth sensor can be, for example, a sensor that performs distance measurement using a TOF (Time of Flight) scheme for performing distance measurement using a return time of reflection of pulsed light from an object or a structured light scheme for irradiating lattice-shaped pattern light and performing distance measurement according to distortion of a pattern.

Further, in the light source unit 106, the imaging unit 104 irradiates the imaging target object with light. The light source unit 106 can be realized by, for example, a wide a light emitting diode (LED) for a wide-angle lens. For example, the light source unit 106 may be configured by combining a normal LED and a lens to diffuse light. Furthermore, the light source unit 106 may have a configuration in which light transmitted through an optical fiber (a light guide) is diffused (widened) by a lens. The light source unit 106 may expand the irradiation range by directing the optical fiber itself in a plurality of directions and irradiating the optical fiber with light. Note that, in FIG. 4, the light source unit 106 does not always need to be included in the endoscopic robot arm system 100. A form of the light source unit 106 is not limited if the irradiation light can be guided to the imaging unit 104 supported by the arm unit 102.

(Learning device 200)

The learning device 200 is a device that generates a learning model used when generating autonomous operation control information for causing the endoscopic robot arm system 100 to autonomously operate, for example, with a CPU (Central Processing Unit), an MPU (Micro Processing Unit), or the like. In the embodiment of the present disclosure, a learning model for performing processing corresponding to classification of input information and processing corresponding to a classification result is generated based on characteristics included in various kinds of input information. The learning model may be realized by, for example, a DNN (Deep Neural Network), which is a multilayer neural network having a plurality of nodes including an input layer, a plurality of intermediate layers (hidden layers), and an output layer. For example, in the generation of the learning model, first, various kinds of input information are input via the input layer and extraction processing or the like for characteristics included in the input information is performed in the plurality of intermediate layers connected in series. Subsequently, the learning model can be generated by outputting, via the output layer, as output information corresponding to the input information, various processing results such as a classification result based on the information output by the intermediate layer. However, the embodiment of the present disclosure is not limited thereto.

Note that a detailed configuration of the learning device 200 is explained below. The learning device 200 may be a device integrated with at least one of the endoscopic robot arm system 100, the control device 300, the evaluation device 400, the presentation device 500, and the surgeon side device 600 illustrated in FIG. 4 explained above or may be a separate device. Alternatively, the learning device 200 may be a device provided on the Cloud and communicably connected to the endoscopic robot arm system 100, the control device 300, the evaluation device 400, the presentation device 500, and the surgeon side device 600.

(Control Device 300)

The control device 300 controls driving of the endoscopic robot arm system 100 based on a learning model generated by the learning device 200 explained above. The control device 300 is implemented by, for example, a CPU, an MPU, or the like executing a program (for example, a program according to the embodiment of the present disclosure) stored in a storage unit explained below using a RAM (Random Access Memory) or the like as a work area. The control device 300 is a controller and may be implemented by, for example, an integrated circuit such as an ASIC (Application Specific Integrated Circuit) or an FPGA (Field Programmable Gate Array).

Note that a detailed configuration of the control device 300 is explained below. The control device 300 may be a device integrated with at least any one of the endoscopic robot arm system 100, the learning device 200, the evaluation device 400, the presentation device 500, and the surgeon side device 600 illustrated in FIG. 4 explained above or may be a separate device. Alternatively, the control device 300 may be a device provided on the Cloud and communicably connected to the endoscopic robot arm system 100, the learning device 200, the evaluation device 400, the presentation device 500, and the surgeon side device 600.

(Evaluation Device 400)

The evaluation device 400 evaluates the operation of the endoscopic robot arm system 100 based on a learning model generated by the learning device 200 explained above. The evaluation device 400 is implemented by, for example, a CPU, an MPU, or the like executing a program (for example, a program according to the embodiment of the present disclosure) stored in a storage unit explained below using a RAM or the like as a work area. Note that a detailed configuration of the evaluation device 400 is explained below. The evaluation device 400 may be a device integrated with at least any one of the endoscopic robot arm system 100, the learning device 200, the control device 300, the presentation device 500, and the surgeon side device 600 illustrated in FIG. 4 described above, or may be a separate device. Alternatively, the evaluation device 400 may be a device provided on the Cloud and communicably connected to the endoscopic robot arm system 100, the learning device 200, the control device 300, the presentation device 500, and the surgeon side device 600.

(Presentation Device 500)

The presentation device 500 displays various images. The presentation device 500 displays, for example, an image captured by the imaging unit 104. The presentation device 500 can be, for example, a display including a liquid crystal display (LCD) or an organic EL (Electro-Luminescence) display. Note that the presentation device 500 may be a device integrated with at least any one of the endoscopic robot arm system 100, the learning device 200, the control device 300, the evaluation device 400, and the surgeon side device 600 illustrated in FIG. 4 explained above. Alternatively, the presentation device 500 may be a separate device that is communicably connected, by wire or radio, to at least any one of the endoscopic robot arm system 100, the learning device 200, the control device 300, the evaluation device 400, and the surgeon side device 600.

(Surgeon Side Device 600)

The surgeon side device 600 is a device (a wearable device) set near the surgeon 5067 or worn on the body of the surgeon 5067 and, specifically, can be, for example, a sensor 602 or a user interface (UI) 604.

For example, the sensor 602 can be a sound sensor (not illustrated) that detects uttered voice of the surgeon 5067, a line-of-sight sensor (not illustrated) that detects a line of sight of the surgeon 5067, a motion sensor (not illustrated) that detects a motion of the surgeon 5067, or the like. Here, specifically, the sound sensor can be a sound collection device such as a microphone that can collect uttered voice or the like of the surgeon 5067. The line-of-sight sensor can be an imaging device including a lens and an imaging element. More specifically, the imaging sensor can acquire sensing data including visual line information such as an eye motion, the size of a pupil diameter, and a gaze time of the surgeon 5067.

The motion sensor is a sensor that detects a motion of the surgeon 5067 and, specifically, can be an acceleration sensor (not illustrated), a gyro sensor (not illustrated), or the like. Specifically, the motion sensor detects changes in acceleration, angular velocity, and the like that occur according to the motion of the surgeon 5067 and acquires sensing data indicating the detected changes. More specifically, the motion sensor can acquire sensing data including information such as a head movement, a posture, and a body shake of the surgeon 5067.

The biological information sensor is a sensor that detects biological information of the surgeon 5067 and can be, for example, various sensors that are directly attached to a part of the body of the surgeon 5067 and measure the heartbeat, the pulse, the blood pressure, the brain waves, the respiration, the perspiration, the myoelectric potential, the skin temperature, the skin electrical resistance, and the like of the surgeon 5067. The biological information sensor may include the imaging device (not illustrated) explained above and, in this case, the imaging device may acquire sensing data including information such as the pulse and a movement the facial muscles of expression (facial expression) of the surgeon 5067.

Further, the UI 604 may be an input device that receives an input from the surgeon. Specifically, the UI 604 can be an operation stick (not illustrated), a button (not illustrated), a keyboard (not illustrated), a foot switch (not illustrated), a touch panel (not illustrated), or a master console (not illustrated) that receives a text input from the surgeon 5067 or a sound collection device (not illustrated) that receives voice input from the surgeon 5067.

### 3. Background Leading to Creation of Embodiments of Present Disclosure

Incidentally, in recent years, in the medical observation system 10 explained above, development for causing the endoscopic robot arm system 100 to automatically operate has been advanced. Specifically, an autonomous operation of the endoscopic robot arm system 100 in the medical observation system 10 can be divided into various levels. Examples of the levels include a level at which the surgeon (an operator) 5067 is guided by the system and a level at which a part of operations (tasks) in surgery for, for example, moving the position of the imaging unit 104 and suturing a surgical site is autonomously executed by the system. Examples of the levels further include a level at which operation contents in the surgery is automatically generated by the system and the endoscopic robot arm system 100 performs an operation selected by a doctor from automatically generated operations. In the future, a level at which the endoscopic robot arm system 100 executes all tasks in surgery under monitoring of the doctor or without the monitoring of the doctor.

Note that, in the embodiment of the present disclosure explained below, it is assumed that the endoscopic robot arm system 100 autonomously executes a task (a scope work) of moving the position of the imaging unit 104 on behalf of the scopist and the surgeon 5067 directly performs surgery or performs surgery with remote control with reference to an image captured by the moved imaging unit 104. For example, in endoscopic surgery, an inappropriate scope work leads to an increase in burden on the surgeon 5067 such as fatigue and screen sickness of the surgeon 5067 and, further, there are problems of difficulty in skill itself of the scope work and a shortage of experts. Therefore, there is a strong demand for autonomous scope work by the endoscopic robot arm system 100.

For the autonomous operation of the endoscopic robot arm system 100, it is requested to generate control information (for example, a target value) for the autonomous operation in advance. Therefore, a learning device is caused to perform machine learning of surgical content and the like and data concerning a surgical operation of the surgeon 5067 and an operation of a scope work and the like of the scopist corresponding the surgical content and the like and generate a learning model. Control information is generated with reference to the learning model obtained in this way and a control rule and the like. More specifically, for example, when a conventionally existing autonomous control method for a robot or the like used in a manufacturing line or the like is to be applied to autonomous control of a scope work, a large amount of good operation data (correct data) of the scope work is input to the learning device to cause the learning device to perform machine learning.

However, since preference and a degree of the scope work differ depending on the surgeon 5067 and the like, it is difficult to understand the correct answer. In other words, since the quality of the scope work is related to the sensitivity of a person (the surgeon 5067, the scopist, and the like), there is no suitable method that can quantitatively evaluate goodness of the scope work. Therefore, it is difficult to collect a large amount of operation data of a scope work considered to be good. Even if the learning model can be constructed based on the good operation data of the scope work, it is difficult for the obtained learning model to suitably cover all states (preference of the surgeon 5067, surgical procedure, a condition of an affected part, and the like) because the learning model is constructed by biased operation data because of a small amount of machine-learned data. In other words, it is difficult to appropriately label the scope work because of the nature unique to the scope work. Since it is difficult to collect a large amount of operation data of a good scope work, it is difficult to efficiently construct a learning model concerning the scope work. That is, it is difficult to apply a conventionally existing autonomous control method to the autonomous control of the scope work. In addition, in the medical field, there are restrictions on devices and times that can be used and, further, it is necessary to protect patient privacy. Therefore, it is difficult to obtain a large amount of operation data of the scope work at the time of surgery.

Therefore, in the situation explained above, the present inventors have uniquely conceived to input a large amount of operation data of a bad scope work (which should be avoided) instead of a large amount of operation data (correct answer data) a good scope work to the learning device and cause the learning device to perform machine learning. As explained above, the quality of the scope work is related to the sensitivity of the person. Therefore, when the person is different, the scope work considered to be good is also different. On the other hand, views of a bad scope work (which should be avoided) are common and tend to coincide even if the person is different. Therefore, even when the human sensitivity is considered, it is easier to collect a large amount of data of the bad scope work compared with the good scope work. Therefore, in the embodiment of the present disclosure created by the present inventors, it is possible to efficiently construct a learning model (a learning model for teaching negative cases) considering the human sensitivity by causing the learning device to perform machine learning using a large amount of operation data of the bad scope work. Further, in the present embodiment, a target value is decided to avoid a state (a state that should be avoided) output by the learning model obtained in this way and the autonomous control of the endoscopic robot arm system 100 is performed.

According to the embodiment of the present disclosure created by the present inventors explained above, since a large amount of appropriately labeled data for machine learning can be collected, a learning model can be efficiently constructed.

In the following explanation, “scope work that should be avoided” means a scope work in which an appropriate field of view is not obtained when the surgeon 5067 performs surgery in endoscopic surgery. More specifically, the “scope work that should be avoided” can include, for example, a scope work in which an image and the like of a surgical site or a medical instrument carried by the surgeon 5067 are not obtained. In the present embodiment, the “scope work that should be avoided” is preferably a scope work that is determined to be obviously inappropriate not only for doctors and scopists but also for ordinary people. In the following description, “scope work that may not be avoided” means a scope work obtained by removing the “scope work that should be avoided” explained above from various scope works. In the present specification, “good scope work” means a scope work that is determined to be appropriate by the surgeon and the like. However, as explained above, since the quality of the scope work is related to the human sensitivity, it is assumed that the scope work is not a scope work that is clearly and uniquely determined. Further, in the following explanation, a learning model generated by machine learning the data of the “scope work that should be avoided” is referred to as learning model for teaching negative cases (first learning model).

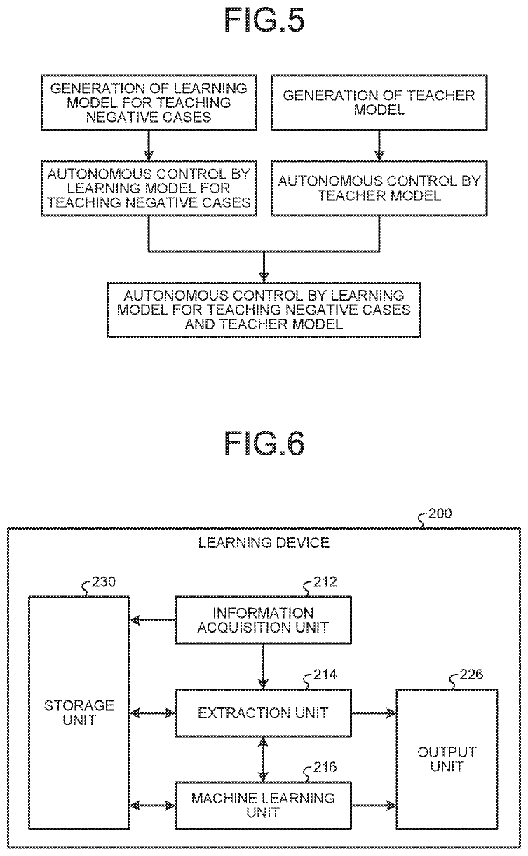

Before details of the embodiments of the present disclosure are explained, an overview of an embodiment of the present disclosure created by the present inventors is explained with reference to FIG. 5. FIG. 5 is an explanatory diagram for explaining an overview of the present embodiment. In the embodiment of the present disclosure explained below, first, as a first embodiment, a learning model for teaching negative cases is generated by performing machine learning of the “scope work that should be avoided” and autonomous control of the endoscopic robot arm system 100 is performed using the generated learning model for teaching negative cases (a flow illustrated on the left side of FIG. 5). As a second embodiment, data of the “scope work that may not be avoided” is collected using a learning model for teaching negative cases, a teacher model (a second learning model) is generated by performing machine learning on the collected data, and autonomous control of the endoscopic robot arm system 100 is performed using the generated teacher model (a flow illustrated on the right side of FIG. 5). As a third embodiment, autonomous control of the endoscopic robot arm system 100 is performed using the learning model for teaching negative cases according to the first embodiment and the teacher model according to the second embodiment (illustrated in the lower part of FIG. 5). Further, in the present disclosure, although not illustrated in FIG. 5, as a fourth embodiment, a scope work of a scopist is evaluated using a learning model for teaching negative cases. Details of such an embodiment of the present disclosure are sequentially explained below.

### 4. First Embodiment

<4.1 Generation of a Learning Model for Teaching Negative Cases>

Detailed Configuration of the Learning Device 200

First, a detailed configuration example of the learning device 200 according to the embodiment of the present disclosure is explained with reference to FIG. 6. FIG. 6 is a block diagram illustrating an example of a configuration of the learning device 200 according to the present embodiment. The learning device 200 can generate a learning model for teaching negative cases used in generating autonomous operation control information. Specifically, as illustrated in FIG. 6, the learning device 200 mainly includes an information acquisition unit (a state information acquisition unit) 212, an extraction unit (a second extraction unit) 214, a machine learning unit (a first machine learning unit) 216, an output unit 226, and a storage unit 230. Details of the functional units of the learning device 200 are sequentially explained below.

(Information Acquisition Unit 212)

The information acquisition unit 212 can acquire various data (state information) concerning a state of the endoscopic robot arm system 100, a state of the surgeon 5067, and the like from the endoscopic robot arm system 100 and the surgeon side device 600 including the sensor 602 and the UI 604 explained above. Further, the information acquisition unit 212 outputs the acquired data to the extraction unit 214 explained later.

In the present embodiment, examples of the data (the state information) include pixel data including image data acquired by the imaging unit 104 and pixel data acquired by a light receiving unit (not illustrated) of a TOF system sensor. In the present embodiment, the data acquired by the information acquisition unit 212 preferably includes at least pixel data such as an image (image data). In the present embodiment, the pixel data is not limited to data acquired when surgery is actually performed and may be, for example, data acquired at the time of simulated surgery using a medical phantom (model) or data acquired by a surgery simulator represented by three-dimensional graphics or the like. Further, in the present embodiment, the pixel data is not necessarily limited to including the data of the medical instrument (not illustrated) or the organ and may include, for example, only the data of the medical instrument or only the data of the organ. In the present embodiment, the image data is not limited to raw data acquired by the imaging unit 104 and may be, for example, data obtained by applying processing (adjustment processing for luminance and saturation, processing for extracting information concerning the position, the posture, and the type of the medical instrument or the organ from an image, semantic segmentation, and the like) to the raw data acquired by the imaging unit 104. In addition, in the present embodiment, information (for example, metadata) such as a recognized or estimated sequence or context of the surgery may be linked with the pixel data.

In the present embodiment, the data (the state information) may be, for example, the positions, the postures, the speeds, the accelerations, and the like of the distal end portion or joint portions (not illustrated) of the arm unit 102 and the imaging unit 104. Such data may be acquired from the endoscopic robot arm system 100 at the time of manual operation or autonomous operation by the scopist or may be acquired from a motion sensor provided in the endoscopic robot arm system 100. Note that, the manual operation of the endoscopic robot arm system 100 may be a method in which the scopist performs an operation on the UI 604 or a method in which the scopist directly physically grips a part of the arm unit 102 and applies a force to the arm unit 102 and, therefore, the arm unit 102 passively operates according to the force. Further, in the present embodiment, the data may be an imaging condition (for example, focus) corresponding to the image acquired by the imaging unit 104. The data may be the type, the position, the posture, the speed, the acceleration, and the like of the medical instrument (not illustrated) supported by the arm unit 102.

Further, the data (the state information) may be, for example, operation information (for example, UI operation) or biological information of the scopist or the surgeon 5067 who manually operates the endoscopic robot arm system 100. More specifically, examples of the biological information include a visual line, blinks, a heartbeat, a pulse, a blood pressure, an electroencephalogram, respiration, sweating, a myoelectric potential, a skin temperature, skin electrical resistance, uttered voice, a posture, and a motion (for example, shaking of the head or the body) of the scopist or the surgeon 5067. For example, when it is determined that the surgeon 5067 or the like falls into a scope work that should be avoided while causing the endoscopic robot arm system 100 to autonomously operate and performing surgery, the surgeon 5067 or the like sometimes performs switch operation and operation for, for example, directly applying force to the arm unit 102, stops the autonomous operation of the endoscopic robot arm system 100, or changes an autonomous operation mode to a manual operation mode. The operation information may include information concerning such operation of the surgeon 5067. For example, when being stored in the storage unit 230 explained below, the operation information is preferably stored in a form in which the data can be explicitly distinguished from other data. Note that the data stored in this manner may include, for example, not only data at an instance when the surgeon 5067 stops the autonomous operation of the endoscopic robot arm system 100 but also data at a transient time to the state (for example, data at time from one second before the autonomous operation is stopped to the stop). The uttered voice can be, for example, uttered voice including negative expression for an endoscopic image such as “this appearance is not good” or “I want you to get closer” uttered by the surgeon 5067 during surgery, that is, uttered voice assumed to be deeply associated with the scope work that should be avoided.

That is, in the present embodiment, it is preferable that the information acquisition unit 212 acquire data without any particular limitation if the data is data serving as a clue for extracting data of an operation of the scope work that should be avoided. Then, in the present embodiment, data of the operation of the scope work that should be avoided is extracted using such data. Therefore, according to the present embodiment, it is possible to extract the data of the operation of the scope work that should be avoided using data that can be naturally acquired without doing anything special while performing surgery using the endoscopic robot arm system 100. Therefore, it is possible to efficiently collect the data.

(Extraction Unit 214)

The extraction unit 214 can extract data labeled as being a predetermined operation from a plurality of data output from the information acquisition unit 212 and output the extracted data to the machine learning unit 216 explained below. More specifically, for example, the extraction unit 214 can extract, using an image analysis or the like, data of an operation of a scope work (for example, a scope work in which a surgical site is not imaged by the imaging unit 104) determined as being an operation that should be avoided from the data acquired when the endoscopic robot arm system 100 is manually operated by the scopist. At this time, the extraction unit 214 may more accurately extract data of an operation of the scope work that should be avoided referring to a stress level, a vital value of sickness or the like of the surgeon 5067, the scopist, or the like obtained by analyzing biological information, words assumed to be deeply associated with the scope work that should be avoided such as “this appearance is not good” obtained by analyzing utterance, or UI operation or the like (for example, emergency stop operation). Further, when information (for example, a time period) correlated with the scope work that should be avoided is known, the extraction unit 214 may extract data of an operation of the scope work that should be avoided referring to such correlated information.

(Machine Learning Unit 216)

The machine learning unit 216 can perform machine learning on the data of the operation of the scope work that should be avoided (a plurality of state information concerning the operation of a medical arm labeled as being an operation that should to be avoided) output from the extraction unit 214 and generate a learning model for teaching negative cases. The learning model for teaching negative cases is used when the control device 300 explained below controls the endoscopic robot arm system 100 to autonomously operate to avoid a state output from the learning model for teaching negative cases. Then, the machine learning unit 216 outputs the generated learning model for teaching negative cases to the output unit 226 and the storage unit 230 explained below. Note that, in the present embodiment, the machine learning unit 216 can also perform machine learning using a plurality of data of different types (for example, positions, postures, and speeds) labeled as being an operation that should be avoided and can further perform machine learning using a plurality of data of the same type and in different states labeled as being the operation that should be avoided.

More specifically, the machine learning unit 216 is assumed to be, for example, a supervised learning device such as a support vector regression or a deep neural network (DNN). For example, the machine learning unit 216 can acquire feature values (for example, feature values about the position, the posture, the speed, the acceleration, and the like of the arm unit 102 and the imaging unit 104, a feature value about an image acquired by the imaging unit 104, and a feature value about an imaging condition corresponding to the image) characterizing the operation of the scope work that should be avoided by performing a multivariate analysis on data of the operation of the scope work that should be avoided and generate, from the current state concerning the acquired feature values, a learning model for teaching negative cases indicating a correlation with a state assumed next in the case of the scope work that should be avoided. Therefore, by using such a learning model for teaching negative cases, for example, in the case of the scope work that should be avoided, it is possible to estimate, from the current state, pixel data such as an image acquired by the imaging unit 104, states such as the positions, the postures, the speeds, and the accelerations of the distal end portion or the joint portions (not illustrated) of the arm unit 102 and the imaging unit 104, and a state (a feature value) of the image, which could occur next.

As a specific example, the machine learning unit 216 can perform machine learning using data at time t+Δt as teacher data and using the data at time t as input data. In the present embodiment, the machine learning unit 216 may use a formula-based algorithm such as the Gaussian Process Regression model that can be treated more analytically or may be a semi-supervised learning device or a weakly supervised learning device and is not particularly limited.

(Output Unit 226)

The output unit 226 can output the learning model for teaching negative cases, which is output from the machine learning unit 216, to the control device 300 and the evaluation device 400 explained below.

(Storage Unit 230)

The storage unit 230 can store various kinds of information. The storage unit 230 is realized by, for example, a semiconductor memory element such as a RAM (Random Access Memory) or a flash memory or a storage device such as a hard disk or an optical disk.

Note that, in the present embodiment, the detailed configuration of the learning device 200 is not limited to the configuration illustrated in FIG. 6. In the present embodiment, the learning device 200 may include, for example, a recognition unit (not illustrated) that recognizes, from a plurality of data output from the information acquisition unit 212, by using, for example, an image analysis, the type, the position, the posture, and the like of a medical instrument (not illustrated) used by the surgeon 5067. Further, the learning device 200 may include, for example, a recognition unit (not illustrated) that recognizes, from the plurality of data output from the information acquisition unit 212, by using, for example, an image analysis, the type, the position, the posture, and the like of an organ of a surgical site to be treated by the surgeon 5067.

A method for generating a learning model for teaching negative cases

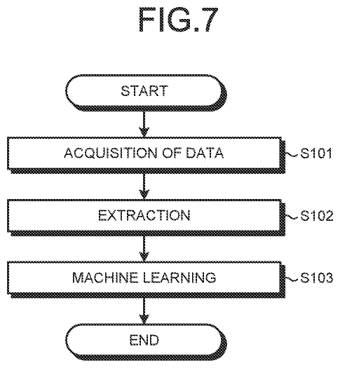

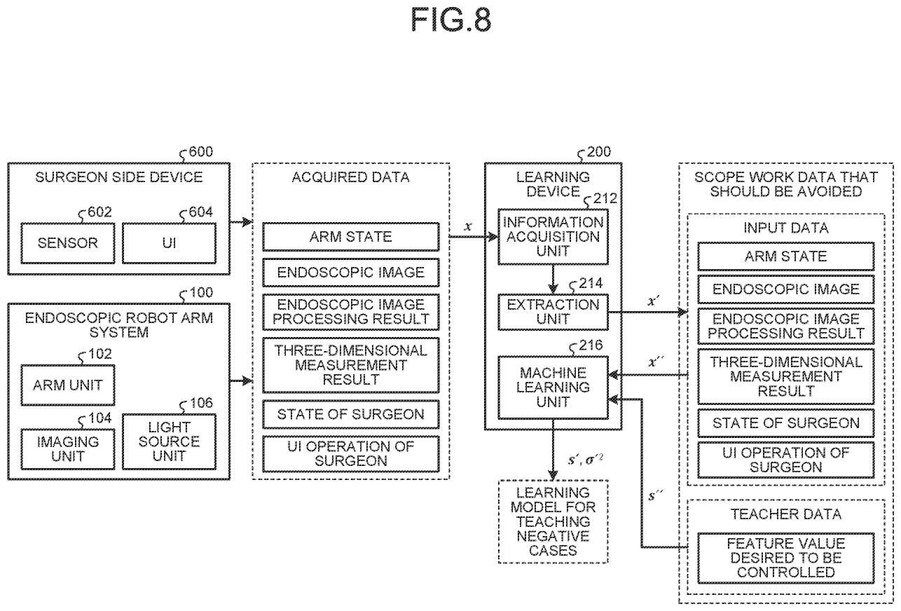

Subsequently, a method for generating a learning model for teaching negative cases according to the present embodiment is explained with reference to FIG. 7 and FIG. 8. FIG. 7 is a flowchart illustrating an example of a method of generating a learning model for teaching negative cases according to the present embodiment. FIG. 8 is an explanatory diagram illustrating an example of the method of generating a learning model for teaching negative cases according to the present embodiment. Specifically, as illustrated in FIG. 7, the method for generating a learning model for teaching negative cases according to the present embodiment includes a plurality of steps from step S101 to step S103. Details of these steps according to the present embodiment are explained below.

First, as illustrated in FIG. 8, the learning device 200 acquires various data concerning a state of the endoscopic robot arm system 100, a state of the surgeon 5067, and the like as a data set x from the endoscopic robot arm system 100 and the surgeon side device 600 including the sensor 602 and the UI 604 (step S101).

Subsequently, the learning device 200 extracts data x′ of the operation of the scope work that should be avoided (for example, a scope work in which the surgical site is not imaged by the imaging unit 104) from the data x acquired when the endoscopic robot arm system 100 is manually operated by the scopist (step S102). For example, when the surgeon 5067 or the like confirms an image captured by the imaging unit 104 and determines that a scope work is the scope work that should be avoided, the data x′ related to the scope work may be extracted by designating the scope work with manual operation. The learning device 200 may extract, based on information considered to have a correlation with the scope work that should be avoided (for example, a head movement and a heart rate of the surgeon 5067), as the operation data x′ of the scope work that should be avoided, data acquired simultaneously with certain information between relevant layers. Note that, in the present embodiment, the learning device 200 not only extracts the data x′ of the operation of the scope work that should be avoided but also may extract data in a transient time period before reaching the scope work. By doing so, in the present embodiment, even in a situation where a scope work is not bad, it is possible to predict, with a learning model, a bad state (a scope work that should be avoided) in which the surgeon 5067 or the like could fall in the future from the situation.

Then, the learning device 200 performs supervised machine learning using the data x′ of the operation of the scope work that should be avoided and generates a learning model for teaching negative cases (step S103). Specifically, in the present embodiment, the control device 300 explained below controls the endoscopic robot arm system 100 to avoid a state output based on the learning model for teaching negative cases. In the present embodiment, the learning model for teaching negative cases is set according to a feature value focused at the time of control of the endoscopic robot arm system 100. A vector representing, as a feature value, a state of the operation of the scope work that should be avoided is explained as s″.

For example, as an example, a case is explained in which the endoscopic robot arm system 100 is autonomously controlled by an algorithm for setting the distal end position of a medical instrument (not illustrated) carried by the right hand of the surgeon 5067 to be the center of a screen and moving the distance between the imaging unit 104 and the medical instrument to a predetermined distance. In this case, the teacher data s″ acquired from the data x′ of the operation of the scope work that should be avoided can be a position coordinate of the distal end of the medical instrument carried by the right hand and distance information between the imaging unit 104 and the medical instrument, the position coordinate and the distance information being arranged to be a vector. More specifically, as illustrated in FIG. 8, a combination of input data x″, which is only data used for learning extracted from the operation data x′ of the scope work that should be avoided, and the teacher data s″ can be, for example, the following data.

Teaching data: a combination of the coordinate on the screen of the distal end of the medical instrument carried by the right hand of the surgeon 5067, the distance information between the imaging unit 104 and the medical instrument, and information indicating a type of the medical instrument at time t+Δt (=s″(t+Δt))

Input data: a combination of a coordinate on the screen of the distal end of the medical instrument carried by the right hand of the surgeon 5067, distance information between the imaging unit 104 and the medical instrument, and information indicating a type of the medical instrument at time t (=x″(t))

Here, Δt is a time width. At may be a sampling time width of acquired data or may be a time longer than the sampling time width. Further, in the present embodiment, the teacher data and the input data are not necessarily limited to be data having a chronological anteroposterior relationship. In the present embodiment, the teacher data s″ is selected according to the feature value focused at the time of the control of the endoscopic robot arm system 100. However, concerning the input data x″, not only the data of the operation of the scope work that should be avoided but also other related data such as biological information of the surgeon 5067 may be flexibly added.

Subsequently, an example of a specific method in which the learning device 200 generates a learning model from the teacher data s″ and the input data x″ is explained. Here, it is assumed that the number of data points acquired so far is N and, when n is 1≤n≤N, an nth data point is represented as s″ₙ and x″ₙ. When an i-th component of s″ₙ is represented as s″ₙᵢ, a vector tᵢ can be represented by the following Expression (1).

tᵢ=[s″₁ᵢ,s″₂ᵢ, . . . ,s″^{Ni}]^{T} (1)

When new input data x″_{N+1} is given based on the Gaussian process regression model, an expected value s′ᵢ of an i-th element of an estimated value s′ of a state of the operation of the scope work that should be avoided and variance σ′² corresponding to the estimated value s′ can be represented by the following Expression (2).

s′ᵢ=k^{T}C_{N}⁻¹tᵢ

σ′²=c−k^{T}C_{N}⁻¹k (2)

Here, C_{N} is a covariance matrix and an n-th row x m-th column element C_{Nnn} is represented by the following Expression (3).

C_{Nnm}(xₙ,xₘ)=k(xₙ,xₘ)+β⁻¹δₙₘ (3)

Further, k in Expression (3) is a kernel function and only has to be selected such that the covariance matrix CN given by Expression (3) is a positive constant. More specifically, k can be given by, for example, the following Expression (4).

$$k ( {x}_{n} , {x}_{m} ) = {θ}_{0} exp \{ - \frac{{θ}_{1}}{2} {‖ {x}_{n} - {x}_{m} ‖}^{2} \} + {θ}_{2} + {θ}_{3} {x}_{n}^{T} {x}_{m} \qquad ( 4 )$$

Note that, in Expression (4), θ₀, θ₁, θ₂, and θ₃ are adjustable parameters.

β in Expression (3) is a parameter representing accuracy (the inverse of variance) in the case in which noise superimposed at the time of observation of s″ₙᵢ follows the Gaussian distribution. δₙₘ in Expression (3) is Kronecker delta.

Further, c in Expression (2) can be represented by the following Expression (5).

c=k(xₙ,xₙ₊₁)+β⁻¹ (5)

It can be said that k in Expression (2) is a vector having k(xₙ, x_{N+1}) as an n-th element.

According to the algorithm explained above, in the present embodiment, the learning device 200 can obtain a learning model for teaching negative cases that can output the estimated value s′ and the variance σ′² of the state of the operation of the scope work that should be avoided. Here, the variance σ′² can be variance indicating the accuracy of the estimated value s′ of the state of the operation of the scope work that should be avoided.

As explained above, in the present embodiment, it is possible to generate, based on the data of the operation of the scope work that should be avoided, a learning model for teaching negative cases that can output the state of the operation of the scope work that should be avoided. As explained above, the views of the scope work that should be avoided are common and tend to coincide even if a person is different. Therefore, in the present embodiment, it is possible to efficiently collect a large amount of data of the operation of the scope work that should be avoided and efficiently construct a learning model for teaching negative cases considering the human sensitivity.

<4.2 Autonomous Control by a Learning Model for Teaching Negative Cases>

Detailed Configuration of the Control Device 300

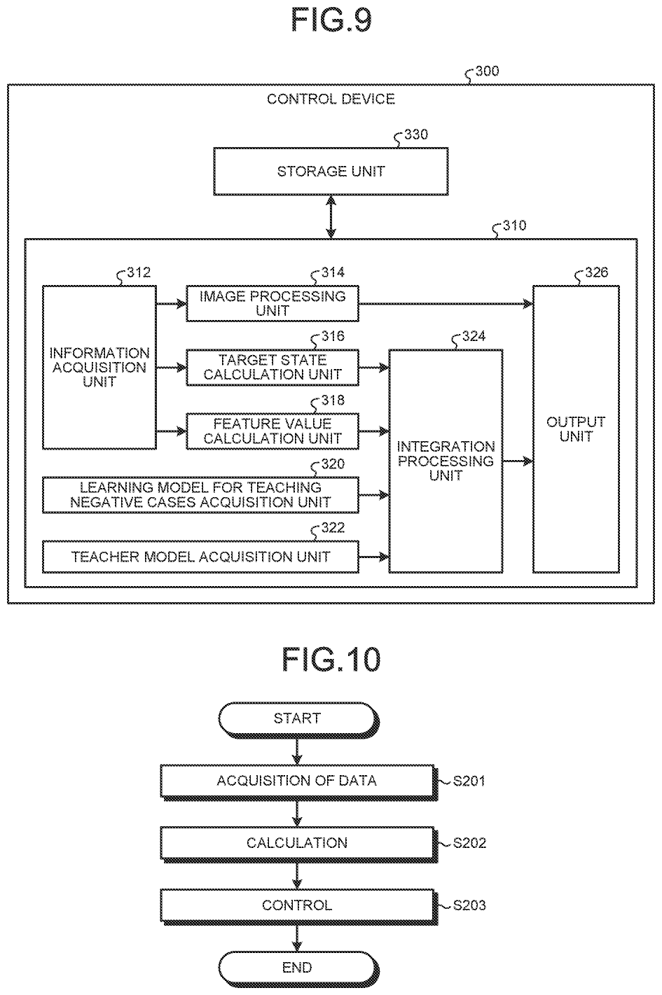

First, a detailed configuration example of the control device 300 according to the embodiment of the present disclosure is explained with reference to FIG. 9. FIG. 9 is a block diagram illustrating an example of a configuration of the control device 300 according to the present embodiment. The control device 300 can autonomously control the endoscopic robot arm system 100 using a learning model for teaching negative cases. Specifically, as illustrated in FIG. 9, the control device 300 mainly includes a processing unit 310 and a storage unit 330. Details of the functional units of the control device 300 are sequentially explained.

(Processing Unit 310)

As illustrated in FIG. 9, the processing unit 310 mainly includes an information acquisition unit 312, an image processing unit 314, a target state calculation unit (an operation target determination unit) 316, a feature value calculation unit 318, a learning model for teaching negative cases acquisition unit 320, a teacher model acquisition unit 322, an integration processing unit (a control unit) 324, and an output unit 326.

The information acquisition unit 312 can acquire various data concerning a state of the endoscopic robot arm system 100, a state of the surgeon 5067, and the like from the endoscopic robot arm system 100 and the surgeon side device 600 including the sensor 602 and the UI 604 explained above in real time during the operation of the endoscopic robot arm system 100. In the present embodiment, examples of the data include pixel data such as an image acquired by the imaging unit 104, the positions, the postures, the speeds, the accelerations, and the like of the distal end portion and the joint portions (not illustrated) of the arm unit 102 and the imaging unit 104, imaging conditions corresponding to an image acquired by the imaging unit 104, the type, the position, the posture, the speed, the acceleration, and the like of the medical instrument (not illustrated) supported by the arm unit 102, operation information (for example, a UI operation) and biological information of the scopist or the surgeon 5067, and the like. For example, the data acquired by the information acquisition unit 312 is not limited to all of the data acquired as explained above and may be an image currently acquired by the imaging unit 104, data obtained by processing the image, or only the position, the posture, the speed, the acceleration, and the like of the distal end portion or the joint portions of the arm unit 102. Further, the information acquisition unit 312 outputs the acquired data to the image processing unit 314, the target state calculation unit 316, and the feature value calculation unit 318 explained below.

The image processing unit 314 can execute various kinds of processes on the image captured by the imaging unit 104. Specifically, for example, the image processing unit 314 may generate a new image by cutting out and enlarging a display target area in the image captured by the imaging unit 104. The generated image is output to presentation device 500 via the output unit 326 explained below.

Further, the processing unit 310 includes a target state calculation unit 316 and a feature value calculation unit 318 that determine an operation target of the endoscopic robot arm system (a medical arm) 100. The target state calculation unit 316 can calculate a target value s* of a feature value desired to be controlled, which should be present at the next moment, and output the target value s* to the integration processing unit 324 explained below. For example, the target state calculation unit 316 calculates, as the target value s*, according to, for example, a combination of medical instruments (not illustrated) present in the field of view of the imaging unit 104, based on a predetermined rule, a state in which the distal end of a predetermined medical instrument is located in the center of the field of view. Alternatively, the target state calculation unit 316 may analyze a motion or the like of the surgeon 5067 and set, as the target value s*, a position where medical instruments carried by the left hand of the right hand of the surgeon 5067 can be appropriately imaged by the imaging unit 104. Note that, in the present embodiment, the algorithm of the target state calculation unit 316 is not particularly limited and may be a rule base based on knowledge obtained so far, a learning base, or a combination the rule base and the learning base. In the present embodiment, it is assumed that the target value s* is likely to include the state of the operation of the scope work that should be avoided.

The feature value calculation unit 318 can extract, from the data output from the information acquisition unit 312, the current state s of the feature value that should be controlled and output the current state s to the integration processing unit 324 explained below. For example, when it is attempted to control the position of the distal end of a medical instrument (not illustrated) carried by the right hand of the surgeon 5067 and the distance of the medical instrument on an image, the feature value calculation unit 318 extracts data concerning the position and the distance from the data output from the information acquisition unit 312, performs calculation, and sets the data as the feature value s. Note that, in the present embodiment, a type of the feature value s is required to be set the same as the target value s* calculated by the target state calculation unit 316 explained above.

The learning model for teaching negative cases acquisition unit 320 can acquire a learning model for teaching negative cases from the learning device 200 and output the learning model for teaching negative cases to the integration processing unit 324 explained below. The teacher model acquisition unit 322 can also acquire a teacher model from the learning device 200 and output the teacher model to the integration processing unit 324 explained below. A detailed operation of the teacher model acquisition unit 322 is explained in a second embodiment of the present disclosure explained below.

The integration processing unit 324 can control driving of the arm unit 102 including the joint portions and ring portions (for example, the integration processing unit 324 controls rotating speeds of motors by controlling an amount of current supplied to the motors in the actuators of the joint portions and controls rotation angles and generated torques in the joint portions), control imaging conditions (For example, focus, a magnification ratio, and the like) of the imaging unit 104, and control the intensity and the like of irradiation light of the light source unit 106. Further, the integration processing unit 324 can autonomously control the endoscopic robot arm system 100 to avoid a state estimated about the learning model for teaching negative cases output from the learning model for teaching negative cases acquisition unit 320. The integration processing unit 324 controls the endoscopic robot arm system 100 to bring the feature value s desired to be controlled close to the operation target (target value s*) determined by the target state calculation unit 316 while performing control to secure a predetermined clearance for the state of the operation of the scope work that should be avoided. More specifically, the integration processing unit 324 finally determines, based on the target value s* and the estimated value s′ of the operation state of the scope work that should be avoided, a control command u given to the endoscopic robot arm system 100. The determined control command u is output to the endoscopic robot arm system 100 via an output unit 326 explained below. At this time, the integration processing unit 324 performs control using, for example, an evaluation function. However, if the accuracy of the estimated value s′ of the operation state of the scope work that should be avoided such as a variance 6′2 explained above can be used as a learning model for teaching negative cases, the evaluation function may be modified according to the accuracy and used.

The output unit 326 can output the image processed by the image processing unit 314 to the presentation device 500 and output the control command u output from the integration processing unit 324 to the endoscopic robot arm system 100.

(Storage Unit 330)

The storage unit 330 can store various kinds of information. The storage unit 330 is realized by, for example, a semiconductor memory element such as a RAM or a flash memory or a storage device such as a hard disk or an optical disk.

Note that, in the present embodiment, the detailed configuration of the control device 300 is not limited to the configuration illustrated in FIG. 9. In the present embodiment, the control device 300 may include, for example, a recognition unit (not illustrated) that recognizes, from a plurality of data output from the information acquisition unit 312, the type, the position, the posture, and the like of a medical instrument (not illustrated) used by the surgeon 5067 by using, for example, an image analysis. Further, the control device 300 may include, for example, a recognition unit (not illustrated) that recognizes, from a plurality of data output from the information acquisition unit 312, the type, the position, the posture, and the like of an organ of a surgical site treated by the surgeon 5067 by using, for example, an image analysis or the like.

Control Method

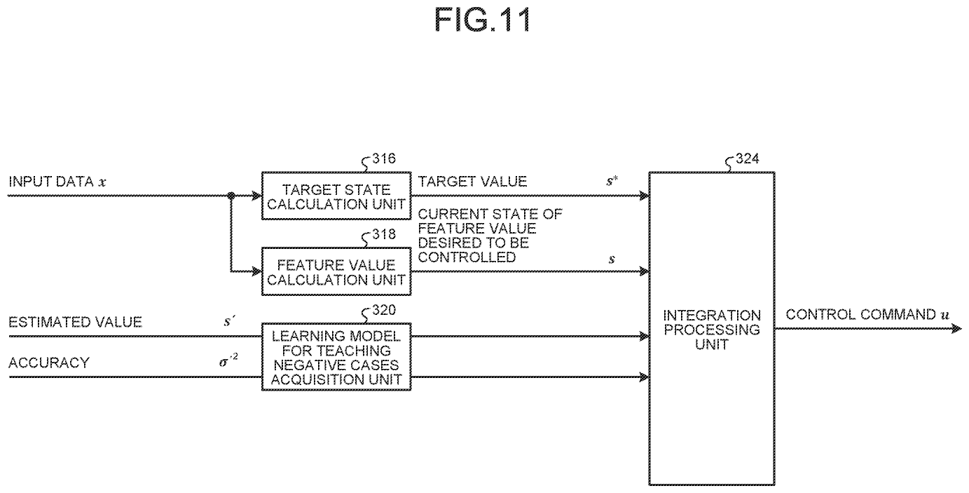

Subsequently, a control method according to the present embodiment is explained with reference to FIG. 10 and FIG. 11. FIG. 10 is a flowchart illustrating an example of a control method according to the present embodiment. FIG. 11 is an explanatory diagram for explaining the control method according to the present embodiment. As illustrated in FIG. 10, the control method according to the present embodiment can include a plurality of steps from step S201 to step S203. Details of these steps according to the present embodiment are explained below.

The control device 300 acquires various data concerning a state of the endoscopic robot arm system 100, a state of the surgeon 5067, and the like in real time from the endoscopic robot arm system 100 and the surgeon side device 600 including the sensor 602 and the UI 604 (step S201).

The control device 300 calculates the control command u (step S202). An example of a specific calculation method at this time is explained below.

For example, an image output of the imaging unit 104 is represented as m, a parameter concerning an object such as an imaging condition and a known size and shape of the object is represented as a, and a parameter such as a position and a posture of the arm unit 102 of the endoscopic robot arm system 100 is represented as q. Note that, as q, time differentiation of the position, posture, and the like of the arm unit 102 may also be included in elements according to necessity. As q, an element of an optical or electronic state quantity such as zoom amount adjustment of the imaging unit 104 or cut-out of a specific region of an image may also be included. On such a premise, a control deviation e at the time when the control system of the endoscopic robot arm system 100 is controlled to converge to 0 can be represented by the following Expression (6).

e=s(q,m,a)−s* (6)

Among the variables for determining the state s desired to be controlled, the q explained above is determined by dynamics of the arm unit 102 and a control input to an actuator mounted on the arm unit 102. In general, q can be represented by a differential equation of the following Expression (7).

\dot{q}=f(q,u) (7)

A function f in Expression (7) only has be set to represent an appropriate robot model according to the idea of control system design. For example, a non-linear motion equation derived from the theory of dynamics of a robot arm is applied as the function f. The function f can be considered torque generated in the actuators disposed in the joint portions (not illustrated) when the control command u is transmitted to the arm unit 102. An equation obtained by linearizing the nonlinear equation of motion can also be applied to the function f according to necessity.

It is not always necessary to apply the motion equation of the robot itself to the function f. Dynamics controlled by the motion control system of the robot may be applied to the function f. As a specific example, since the imaging unit 104 is inserted into the body through a trocar provided in the abdomen of the patient, it is appropriate that the arm unit 102 supporting the imaging unit 104 is controlled such that the imaging unit 104 receives an imaginary constraint (plane two degrees of freedom constraint at one point on the abdominal wall) with the trocar. Therefore, as the function f, dynamics reflecting the fact that the imaging unit 104 located at the distal end of the arm unit 102 is restrained on the trocar and response speed such as insertion and removal and posture change of the imaging unit 104 is artificially set by the control system may be mathematically modeled and used. At this time, the control command u is not necessarily torque generated by the actuator of the arm unit 102 and may be a new control input artificially set by the motion control system. For example, when the motion control system is configured to receive a movement amount of the visual field of the imaging unit 104 as a command and then determine the torques of the joint portions (not illustrated) of the arm unit 102 necessary for realizing the command, the control command u can be considered as the movement amount of the visual field.

Subsequently, the control device 300 controls the endoscopic robot arm system 100 (step S203). Here, as the control of the endoscopic robot arm system 100, an example of a control algorithm for bringing the state s at the present point in time close to the target value s* is explained. Subsequently, an example of a control algorithm for avoiding the estimated value s′ of the state of the operation of the scope work that should be avoided output by the learning model for teaching negative cases is explained.

Example of the control algorithm for bringing the state s close to the target value s*

The control can be grasped as a kind of an optimization problem for, while searching for the state q of the arm unit 102 in which an evaluation function V of the following Expression (8) is minimized, calculating a control command u for converging the state of the arm unit 102 to q.

V=½e(q)^{T}Q_{V}e(q) (8)

Note that, in Expression (8), Q_{V} is a weight matrix. However, q and u cannot be freely determined. At least Expression (7) explained above is applied as a constraint condition.

As a method for solving such an optimization problem, there is model predictive control as a solution practically used in the field of control theory. The model predictive control is a method of performing feedback control by numerically solving an optimal control problem in a finite time interval in real time and is called receding horizon control as well.

Therefore, when the evaluation function is rewritten in a form to which the model predictive control can be applied, Expression (8) described above can be represented by the following Expression (9).

J=φ(qₘ(t+T))+∫ₜ^{t+T}L(qₘ(τ),uₘ(τ))dτ

L=½[s(qₘ(t))−s*]^{T}Q[s(qₘ(t))−s*]+½uₘ(t)^{T}Ruₘ(t)φ(qₘ(t))=[s(qₘ(t))−s*]^{T}Q_{fin}[s(qₘ(t))−s*] (9)

A constraint condition is represented by the following Expression (10).

\dot{q}ₘ=f(qₘ,uₘ)

qₘ(t)=q(t)(10)

In Expression (9) and Expression (10), Q, R, and Q_{fin} are weight matrices and a function ϕ represents terminal cost. In the expressions, qₙ (τ) and uₘ (τ) are merely a state and a control input for executing an operation of the model predictive control and do not necessarily coincide with a state and a control input of an actual system. However, the lower expression of Expression (10) is established only at the initial time.

As an optimization algorithm for calculating control input u*ₘ(τ) and (t≤τ≤t+T) for minimizing J in real time, for example, a GMRES (Generalized Minimal Residual) method considered to be suitable for model predictive control can be used. In this way, an actual control command u(t) actually given to the arm unit 102 at the time t can be determined by the following Expression (11) using, for example, only a value at the time t.

u(t)=u*ₘ(t) (11)

Example of a control algorithm for avoiding the estimated value s′ of the state of the operation of the scope work that should be avoided output by the learning model for teaching negative cases

Subsequently, an example of a control algorithm for avoiding the estimated value s′ of the state of the operation of the scope work that should be avoided output based on the learning model for teaching negative cases is explained. In order to realize such control, for example, the control algorithm for bringing the state s close to the target value s* explained only has to be expanded above such that the value of the evaluation function increases when the state s approaches the value of the estimated value s′. Specifically, an evaluation function L shown in the middle part of Expression (9) can be realized by rewriting Expression (9) to the following Expression (12).

$${L}^{′} = L + P ( s ( {q}_{m} ) ) \qquad ( 12 )$$

$$P ( s ( {q}_{m} ) ) = \frac{K}{{[ s ( {q}_{m} ( t ) ) - {s}^{′} ]}^{T} [ s ( {q}_{m} ( t ) ) - {s}^{′} ]}$$

A function P in Expression (12) is a so-called penalty function in the optimization theory and K is a gain for adjusting an effect of penalty. In this way, in the present embodiment, as illustrated in FIG. 11, in a process of the control for converging the state s to the target value s*, it is possible to control the state s not to approach, as much as possible, the estimated value s′ of the state of the operation state of the scope work that should be avoided.

Note that, in the control using the estimated value s′ of the state of the operation of the scope work that should be avoided output based on the learning model for teaching negative cases, when the current state information x of the endoscopic robot arm system 100 and the input data x″ used when learning the learning model for teaching negative cases are greatly different from each other, there is a possibility that the endoscopic robot arm system 100 is controlled in an unexpected direction and cannot be suitably controlled. Therefore, in the present embodiment, it is preferable to, considering such a case, perform control such that the accuracy σ′² of the estimated value s′ is used as well. For example, in the Gaussian process regression model explained above, the learning device 200 can also output the variance σ′² in addition to the expected value (the estimated value) s′. In addition, as explained above, when the variance σ′² is large, this means that the accuracy of the expected value (the estimated value) s′ is low. Therefore, in the present embodiment, for example, when the variance σ′² is larger than a predetermined value, control may be performed to ignore a penalty term of the evaluation function L′ (Expression 12). Alternatively, in the present embodiment, a gain K of the penalty term of the evaluation function L′ may be defined to depend on the variance σ′². More specifically, by reducing the gain K when the variance σ′² is large, when the accuracy is low, control may be performed not to automatically consider the estimated value s′ of the state of the operation of the scope work that should be avoided output by the learning model for teaching negative cases. Note that, in the present embodiment, besides such a method, various methods for solving the optimization problem with the constraint condition such as a barrier method and a multiplier method may be applied.

As explained above, in the present embodiment, it is possible to control the endoscopic robot arm system 100 to avoid the estimated value s′ in the state of the operation of the scope work that should be avoided, which is output based on the learning model for teaching negative cases based on the data of the operation of the scope work that should be avoided. Therefore, according to the present embodiment, since the learning model for teaching negative cases can be used considering the human sensitivity and sensory aspect hard to be handled by a mathematical approach, it is possible to autonomously control the endoscopic robot arm system 100 considering the human sensitivity and the like.

### 5. Second Embodiment

In a second embodiment of the present disclosure explained next, data of “scope work that may not be avoided” is collected using the learning model for teaching negative cases explained above and a teacher model is generated by performing machine learning on the collected data. In the present embodiment, autonomous control of the endoscopic robot arm system 100 is performed using the generated teacher model.

<5.1 Generation of a Teacher Model>

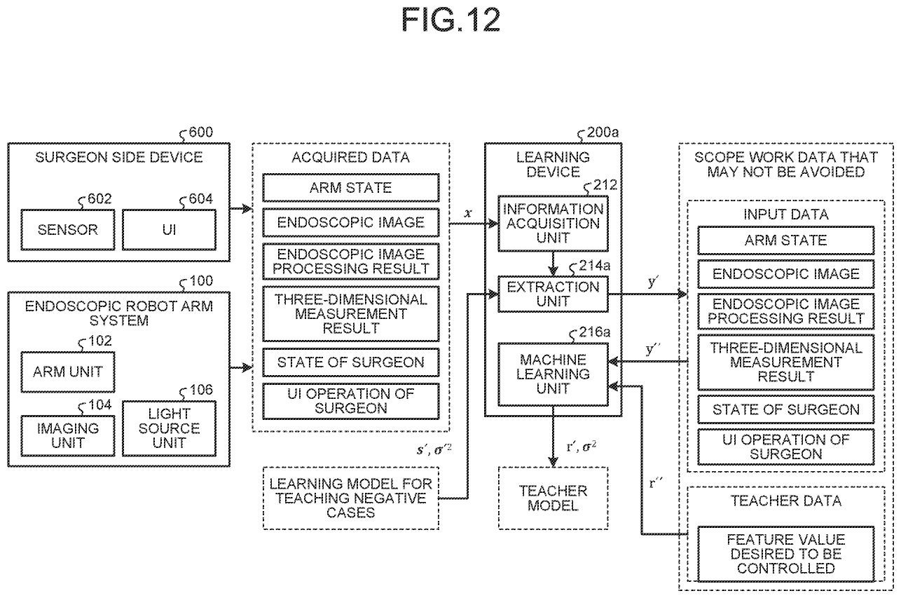

Detailed Configuration of a learning device 200a First, a detailed configuration example of the learning device 200a according to the present embodiment is explained with reference to FIG. 12. FIG. 12 is an explanatory diagram for explaining a method of generating a teacher model according to the present embodiment. The learning device 200a can generate a teacher model used in generating autonomous operation control information. Specifically, as illustrated in FIG. 12, the learning device 200a mainly includes an information acquisition unit (a state information acquisition unit) 212, an extraction unit (a first extraction unit) 214a, a machine learning unit (a second machine learning unit) 216a, an output unit 226 (not illustrated in FIG. 12), and a storage unit 230 (not illustrated in FIG. 12). Details of the functional units of the learning device 200a are sequentially explained below. Note that, in the present embodiment, since the information acquisition unit 212, the output unit 226, and the storage unit 230 are common to the first embodiment, explanation of the units is omitted here.

(Extraction Unit 214 a)

The extraction unit 214a can extract, from the data (the state information) x acquired when the endoscopic robot arm system 100 is manually operated by the scopist, data (state information labeled as being an operation that may not be avoided) y′ of an operation of a scope work that may not be avoided (for example, a scope work in which a surgical site is imaged by the imaging unit 104.) based on the learning model for teaching negative cases explained above. Further, the extraction unit 214a can output the extracted data y′ to the machine learning unit 216a explained below. In the related art, the data y′ of the operation of the scope work that may not be avoided can be obtained only by manually removing, from at least a large number of data x, the data x′ of the operation of the scope work that should be avoided. However, in the present embodiment, by using the learning model for teaching negative cases, it is possible to automatically extract the data y′ of the operation of the scope work that may not be avoided. In addition, according to the present embodiment, the teacher model can be generated by using the data y′ obtained in this way. It is possible to improve the accuracy of the autonomous control of the endoscopic robot arm system 100 by using the teacher model.

Here, a specific example of automatically extracting the data y′ of the operation of the scope work that may not be avoided is explained. As illustrated in FIG. 12, the extraction unit 214a acquires a learning model for teaching negative cases (the estimated value s′, variance σ′²) and calculates a difference norm between the state s of a large number of data and the estimated value s′ as indicated by the following Expression (13). Subsequently, in a case where the difference norm is equal to or smaller than a threshold s_{d}, the extraction unit 214a can automatically extract, by excluding data of the difference norm from the large number of data, the data y′ of the operation of the scope work that may not be avoided.

∥s−s′∥≤s_{d} (13)

Note that, in the present embodiment, as another method, the data y′ of the operation of the scope work that may not be avoided may be automatically extracted using the variance σ′² or the like of the learning model for teaching negative cases.

(Machine Learning Unit 216a)

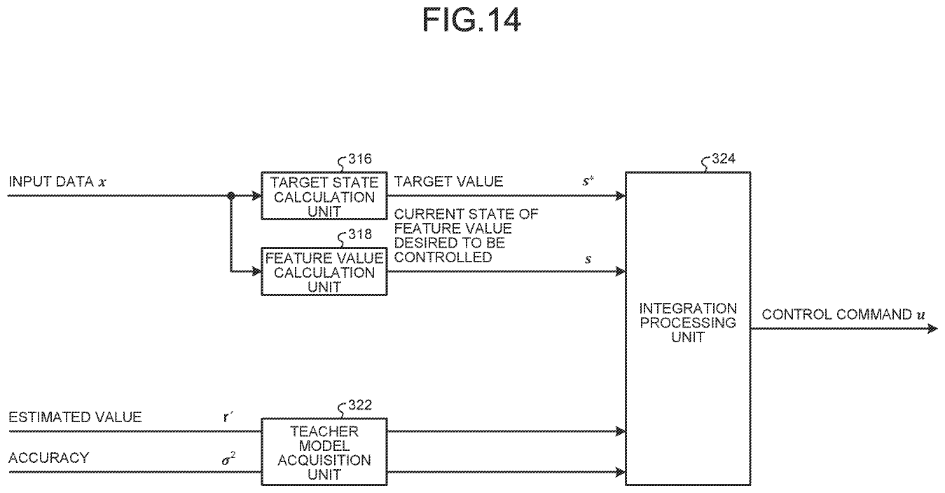

As in the first embodiment, the machine learning unit 216a is a supervised learning device and can generate a teacher model by performing machine learning on data (state information labeled as being an operation that may not be avoided) y″ of the operation of the scope work that may not be avoided output from the extraction unit 214 a. The teacher model is used when the endoscopic robot arm system 100 is controlled to autonomously operate in an integration processing unit 324 (see FIG. 14) of a control device 300a explained below. The machine learning unit 216a outputs the teacher model to the output unit 226 and the storage unit 230.

Note that, in the present embodiment, the detailed configuration of the learning device 200a is not limited to the configuration illustrated in FIG. 12.

Note that, in the present embodiment, since a method of generating a teacher model is common to the first embodiment, explanation of the method of generating a teacher model is omitted here.

<5.2 Autonomous Control by the Teacher Model>

Subsequently, autonomous control of the endoscopic robot arm system 100 using a teacher model is explained. However, since the control device 300 according to the present embodiment is common to the first embodiment, explanation about a detailed configuration example of the control device 300 is omitted here.

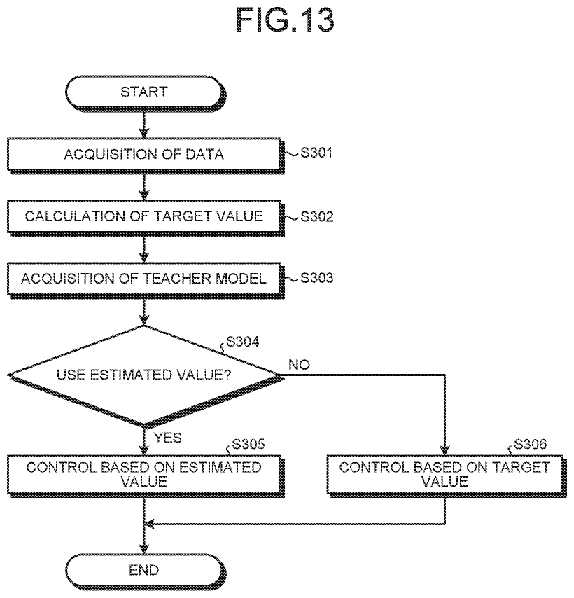

A control method by a teacher model according to the present embodiment is explained with reference to FIG. 13 and FIG. 14. FIG. 13 is a flowchart illustrating an example of the control method according to the present embodiment. FIG. 14 is an explanatory diagram for explaining the control method according to the present embodiment. As illustrated in FIG. 13, the control method according to the present embodiment can include a plurality of steps from step S301 to step S306. Details of these steps according to the present embodiment are explained below.

In the present embodiment, the target value s* is determined considering the estimated value r′ obtained from a teacher model based on the data of the operation of the scope work that may not be avoided and the control command u to the arm unit 102 is determined. Specifically, in the first embodiment, the target value s* is determined based on the rule base such as a mathematical formula. However, in the present embodiment, by using, as the target value s*, the estimated value r′ obtained from the teacher model based on the data of the operation of the scope work that may not be avoided, it is possible to bring the autonomous operation of the endoscopic robot arm system 100 closer to a scope work that further reflects the sensitivity of the surgeon 5067.

However, in the present embodiment, the estimated value r′ obtained from the teacher model based on the data of the operation of the scope work that may not be avoided is not necessarily an estimated value based on data of an operation of a good scope work. Therefore, when control is performed using the estimated value r′ obtained from the teacher model, the endoscopic robot arm system 100 cannot necessarily be suitably autonomously controlled. Therefore, in the present embodiment, as illustrated in FIG. 14, based on a predetermined rule, it is determined which one of the estimated value r′ obtained from the teacher model based on the data of the operation of the scope work that may not be avoided and the target value s* determined by the same method as in the first embodiment is used as a target value of control.

First, as in the first embodiment, the control device 300 acquires various data concerning, for example, a state of the endoscopic robot arm system 100 from the endoscopic robot arm system 100 or the like in real time (step S301). Subsequently, the control device 300 calculates the target value s* as in the first embodiment (step S302). The control device 300 acquires a teacher model from the learning device 200a (step S303).

Subsequently, the control device 300 determines whether to perform control using, as a target value, the estimated value r′ obtained from the teacher model acquired in step S303 (step S304). For example, when the target value s* calculated in step S302 and the estimated value r′ obtained from the teacher model are close, it is estimated that the estimated value r′ obtained from the teacher model does not empirically deviate from a state of an operation of a good scope work assumed in the rule base such as the mathematical formula. Therefore, since the estimated value r′ obtained from the teacher model is highly reliable and is highly likely to be in a state of the scope work reflecting the sense of the surgeon 5067 as well, the estimated value r′ can be used for control as the target value. More specifically, the closeness of the target value s* calculated in step S302 and the estimated value r′ obtained from the teacher model can be determined using the difference norm explained above. In the present embodiment, when the accuracy of the variance 62 or the like obtained from the teacher model is equal to or smaller than a predetermined value, the estimated value r′ obtained from the teacher model may be used for control as the target value.

When determining to perform control using, as the target value, the estimated value r′ obtained from the teacher model acquired in step S303 (step S304: Yes), the control device 300 proceeds to step S305. When determining not to perform control using, as the target value, the estimated value r′ obtained from the teacher model (step S304: No), the control device 300 proceeds to step S306.

The control device 300 controls the endoscopic robot arm system 100 using, as a target value, the estimated value r′ obtained from the teacher model acquired in step S303 (step S305). The control device 300 controls the endoscopic robot arm system 100 using the target value calculated in Step S302 (Step S306). Details of the control method are the same as the details in the first embodiment. Therefore, detailed explanation of the control method is omitted here.

As explained above, in the present embodiment, by using the learning model for teaching negative cases, it is possible to automatically extract the data y′ of the operation of the scope work that may not be avoided. In addition, according to the present embodiment, the teacher model can be generated by using the data y′ obtained in this way. It is possible to improve the accuracy of the autonomous control of the endoscopic robot arm system 100 by using the teacher model.

### 6. Third Embodiment

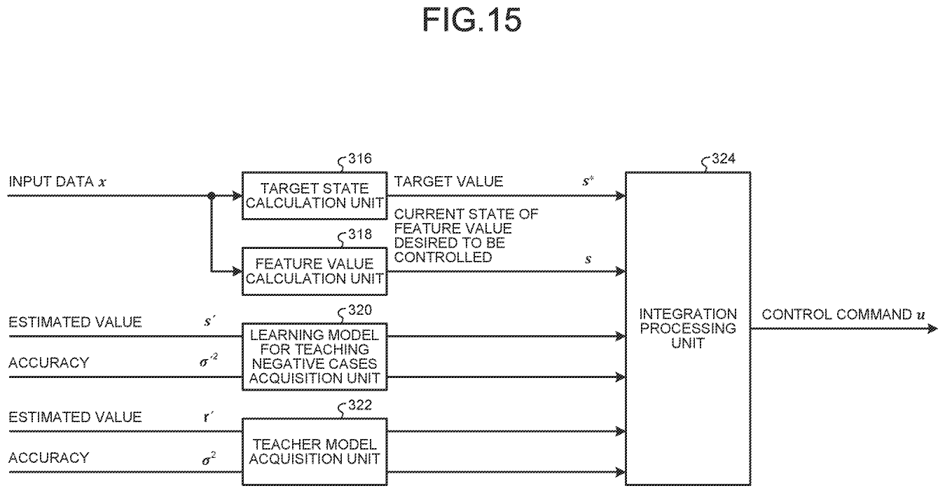

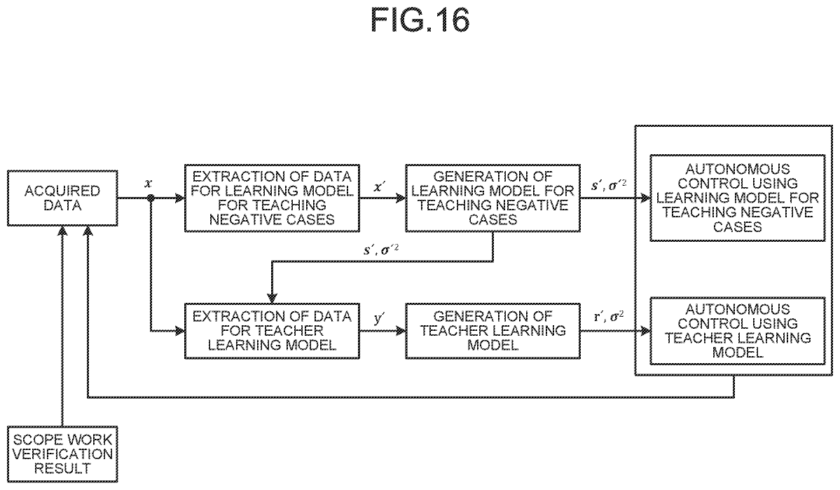

Subsequently, autonomous control of the endoscopic robot arm system 100 using the learning model for teaching negative cases according to the first embodiment and the teacher model according to the second embodiment is explained with reference to FIG. 15 and FIG. 16. FIG. 15 and FIG. 16 are explanatory diagrams for explaining a control method according to the present embodiment. In the present embodiment, by concurrently using the autonomous control using the learning model for teaching negative cases and the autonomous control using the teacher model, it is possible to enjoy the advantages of both of the autonomous controls. Therefore, it is possible to realize autonomous control that reflects the sense of the surgeon 5067 for the scope work hard to be represented by a mathematical formula.

More specifically, in the present embodiment, as illustrated in FIG. 15, as in the first embodiment, the integration processing unit 324 controls the endoscopic robot arm system 100 to avoid the estimated value s′ of the state of the operation of the scope work that should be avoided. At this time, the integration processing unit 324 can control the endoscopic robot arm system 100 using, as the target value, the estimation value r′ obtained from the teacher model based on the data of the operation of the scope work that may not be avoided. Note that, in the present embodiment as well, as in the second embodiment explained above, it is preferable to determine, based on a predetermined rule, which of the estimated value r′ obtained from the teacher model based on the data of the operation of the scope work that may not be avoided and the target value s* determined by the same method as the method in the first embodiment is used as the target value of control. In the present embodiment, the integration processing unit 324 may perform weighting on the estimated value s′ by the learning model for teaching negative cases and the estimated value r′ by the teacher model and control the endoscopic robot arm system 100.

In the present embodiment, first, the endoscopic robot arm system 100 may be controlled to avoid the state of the estimated value s′ by the learning model for teaching negative cases and, then, the endoscopic robot arm system 100 may be controlled to bring the state of the estimated value s′ close to the state of the estimated value r′ by the teacher model. Further, in the present embodiment, the control using the estimated value s′ by the learning model for teaching negative cases and the control using the estimated value r′ by the teacher model may be repeatedly used in a loop shape to control the endoscopic robot arm system 100.

Specifically, as illustrated in FIG. 16, first, the medical observation system 10 according to the present embodiment acquires new data x by executing and verifying autonomous control using the learning model for teaching negative cases (autonomous control using the teacher model may be carried out in parallel). A method of the verification may be performed by the surgeon 5067 himself or herself through surgery on a patient using the endoscopic robot arm system 100 or may be performed using a medical phantom (model) in the endoscopic robot arm system 100. Further, the verification may be performed using a simulator. For example, by using the simulator, a patient, a surgical site, the imaging unit 104, the arm unit 102, a medical instrument, and the like can be virtually reproduced on a virtual space and the surgery can be virtually performed on the surgical site by a doctor. The data x acquired here is a result of performing the autonomous control to avoid a state of an operation of a scope work that should be avoided obtained from at least the learning model for teaching negative cases. However, it is conceivable that the initially obtained data x includes a state of an operation of a scope work that cannot be covered by the learning model for teaching negative cases and should be avoided.

Therefore, in the present embodiment, the control using the estimated value s′ by the learning model for teaching negative cases and the control using the estimated value r′ by the teacher model are repeatedly used in a loop shape. In an initial period of the loop of repetition, since the acquired data x includes a lot of data of the operation of the scope work that should be avoided, it takes time to extract and collect the data of the operation of the scope work that should be avoided. However, by repeating the loop a plurality of times, the learning model for teaching negative cases and the teacher model are matured and the quality of the autonomous control by these models is improved. Therefore, at the same time, the data of the operation of the scope work that should be avoided included in the data x decreases. Therefore, a load of extracting and collecting the data of the operation of the scope work that should be avoided sequentially decreases and improvement of the quality of the learning model for teaching negative cases is promoted. Further, since the quality of the data of the operation of the scope work that may not be avoided is also improved, the quality of the teacher model based on the data of the operation of the scope work that may not be avoided is also improved. Finally, when the learning model for teaching negative cases and the teacher model are more matured, it is possible to extract and collect only data of an operation of a high-quality scope work. Therefore, it is possible to autonomously control the endoscopic robot arm system 100 using only the teacher data based on these data.

Note that the present embodiment is not limited to acquiring the new data x with the verification method explained above and may be, for example, a result obtained by using another learning model or control algorithm or may actually be measurement data of surgery manually performed by the surgeon 5067 and the scopist.

As explained above, according to the present embodiment, on the other hand, by concurrently using the autonomous control using the learning model for teaching negative cases and the autonomous control using the teacher model, it is possible to enjoy the advantages of both the autonomous controls. Therefore, it is possible to realize autonomous control that reflects the sense of the surgeon 5067 for the scope work hard to be represented by a mathematical formula.

### 7. Fourth Embodiment

In the present embodiment, an actual scope work of a scopist is evaluated using the learning model for teaching negative cases explained above and a result of the evaluation is presented to the scopist. In the present embodiment, for example, when the actual scope work is a scope work that should be avoided, the scope work can be notified to the scopist via the notice device 500 or the like. In the present embodiment, the evaluation result can be fed back at the time of training of the scopist (in actual scope work; for example, teaching materials using videos of scope works carried out by other scopists are also included). Therefore, according to the present embodiment, it is possible to promote improvement of the skill of the scopist.

<7.1 Detailed Configuration Example of the Evaluation Device 400>

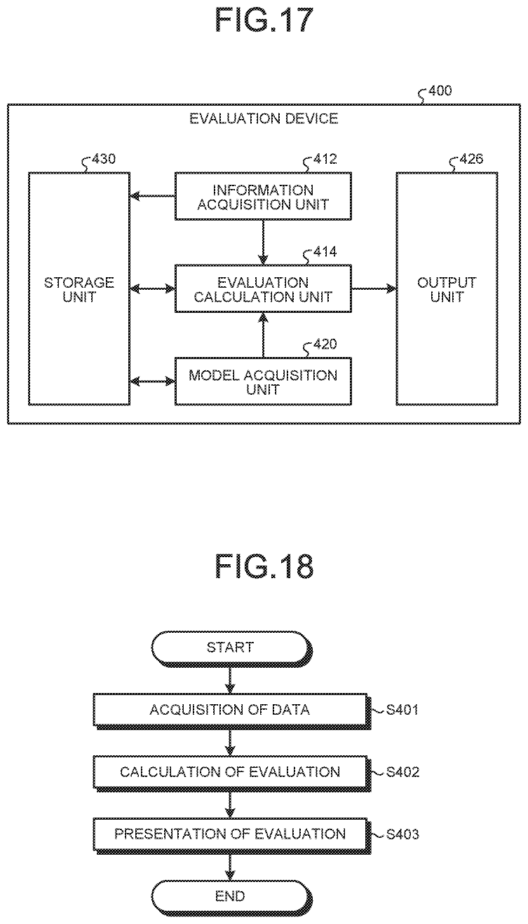

First, a detailed configuration example of the evaluation device 400 according to the embodiment of the present disclosure is explained with reference to FIG. 17. FIG. 17 is a block diagram illustrating an example of a configuration of the evaluation device 400 according to the present embodiment. Specifically, as illustrated in FIG. 17, the evaluation device 400 mainly includes an information acquisition unit 412, an evaluation calculation unit (an evaluation unit) 414, a model acquisition unit 420, an output unit 426, and a storage unit 430. Details of the functional units of the evaluation device 400 are sequentially explained below.

(Information Acquisition Unit 412)

The information acquisition unit 412 can acquire various data concerning a state of the endoscopic robot arm system 100 from the endoscopic robot arm system 100 or the like in real time.

(Evaluation Calculation Unit 414)

The evaluation calculation unit 414 can evaluate a scope work according to a learning model for teaching negative cases (the estimated value s′ and the like) output from the model acquisition unit 420 explained below and output the evaluation result to the output unit 426 to be described later. For example, the evaluation calculation unit 414 calculates, as an evaluation value, norm differences between the state s of feature values at instances and the estimated value s′ of a state of an operation of a scope work that should be avoided obtained from the learning model for teaching negative cases. In this case, it can be interpreted that, as the evaluation value is smaller, the scope work is closer to the scope work that should be avoided.

(Model Acquisition Unit 420)

The model acquisition unit 420 can acquire a learning model for teaching negative cases (the estimated value s′, the variance σ′², and the like) from the learning device 200 and output the learning model for teaching negative cases to the evaluation calculation unit 414.

(Output Unit 426)

The output unit 426 can output the evaluation result from the evaluation calculation unit 414 explained above to the presentation device 500. Note that, in the present embodiment, the evaluation result is not limited to, for example, being displayed by the presentation device 500. For example, as a method of presenting the evaluation result to the scopist in real time, when the evaluation result is in a state worse than a certain index, for example, a wearable device (not illustrated) worn on the scopist may vibrate or output sound or a lamp mounted on the presentation device 500 may blink.

In the present embodiment, instead of presenting the evaluation result in real time, a comprehensive evaluation result may be presented after a series of surgical operations is completed. For example, norm differences between the state s of feature values at instances and the estimated value s′ of the operation of the scope work that should be avoided may be calculated and a time average value the norm differences may be presented as an evaluation result. In this way, in a case where the time average value is high, a notification that the quality of the scope work is low can be presented to the scopist.

(Storage Unit 430)

The storage unit 430 stores various kinds of information. The storage unit 430 is realized by, for example, a semiconductor memory element such as a RAM or a flash memory or a storage device such as a hard disk or an optical disk.

Note that, in the present embodiment, the detailed configuration of the evaluation device 400 is not limited to the configuration illustrated in FIG. 17.

<7.2 Evaluation Method>

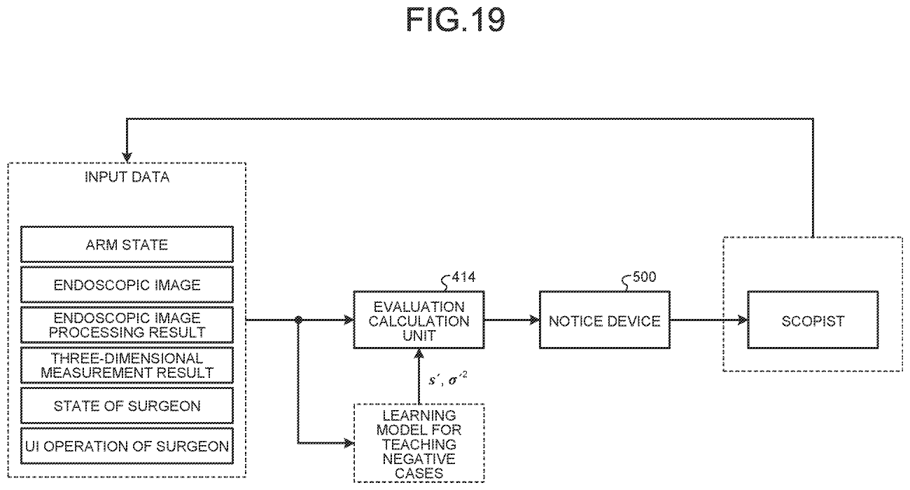

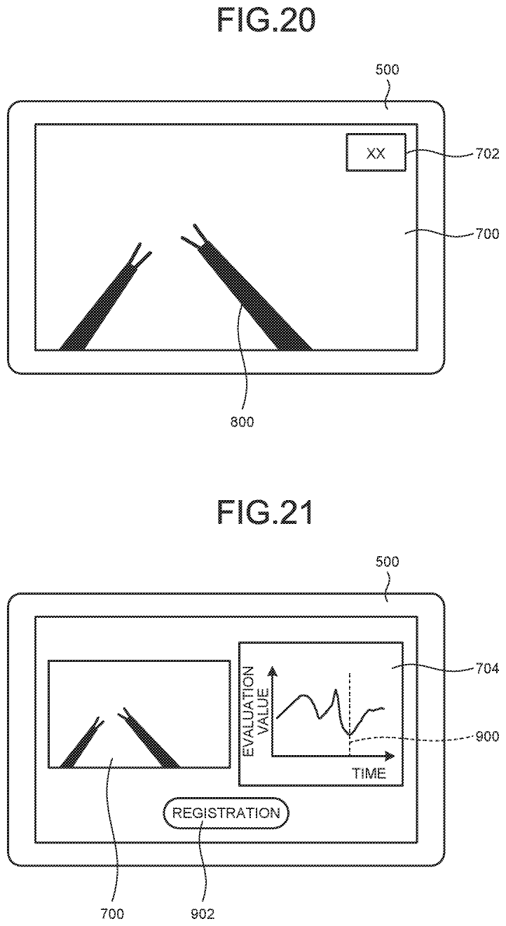

Subsequently, an evaluation method according to the present embodiment is explained with reference to FIG. 18 to FIG. 21. FIG. 18 is a flowchart illustrating an example of the evaluation method according to the present embodiment. FIG. 19 is an explanatory diagram for explaining the evaluation method according to the present embodiment. FIG. 20 and FIG. 21 are explanatory diagrams for explaining an example of a display screen according to the present embodiment. As illustrated in FIG. 18, the evaluation method according to the present embodiment can include a plurality of steps from step S401 to step S403. Details of these steps according to the present embodiment are explained below.

First, the evaluation device 400 acquires various data concerning a state of the endoscopic robot arm system 100 from the endoscopic robot arm system 100 or the like in real time (step S401). Further, as illustrated in FIG. 19, the evaluation device 400 acquires a learning model for teaching negative cases (the estimated value s′, the variance σ′², and the like) from the learning device 200.

Subsequently, as illustrated in FIG. 19, the evaluation device 400 evaluates a scope work based on the data acquired in step S401 according to the learning model for teaching negative cases (the estimated value s′ and the like) and outputs an evaluation result (step S402).

The evaluation device 400 presents the evaluation result to the scopist (step S403). In the present embodiment, for example, when the evaluation result is displayed in real time, as illustrated in FIG. 20, a surgical video 700 including an image of a medical instrument 800 or the like is displayed on a display unit of the presentation device 500. Further, in the present embodiment, the evaluation result is displayed in real time on an evaluation display 702 located at a corner of the display unit not to disturb the scope work of the scopist.

In the present embodiment, for example, when the evaluation result is displayed after the surgery is completed, an evaluation display 704 indicating a time-series change in the evaluation result may be displayed as illustrated in FIG. 21. In this case, in order to temporally synchronize the surgical video 700 and the evaluation result, for example, it is preferable that a user (For example, a scopist) moves the position of a cursor 900 on the evaluation display 704, whereby a video of the surgical video 700 at time corresponding to the position of the cursor 900 is reproduced. Further, in the present embodiment, when it can be determined based on the surgical video 700, the evaluation result, or the like that the scope work related to the surgical video 700 is a scope work that should be avoided, it is preferable that a button 902 for performing operation for registering the surgical video 700 as data of the scope work that should be avoided is displayed on the display unit of the presentation device 500. Note that, in the present embodiment, such registration work may be performed in real time during the surgery or may be performed offline after the surgery.

As explained above, in the present embodiment, the scope work of the scopist can be evaluated using the learning model for teaching negative cases and the evaluation result can be presented to the scopist. Therefore, according to the present embodiment, since it is possible to feed back, as quantitative data, when the scope work of the scopist tends to fall into a bad state, it is possible to utilize the data for training for improving the skill of the scopist.

### 8. Summary

As explained above, according to the embodiment of the present disclosure, it is possible to collect a large amount of appropriately labeled data for machine learning (data of an operation of a scope work that should be avoided and data of an operation of a scope work operation that may not be avoided) and efficiently construct a learning model (a learning model for teaching negative cases or a teacher model).

### 9. Hardware Configuration

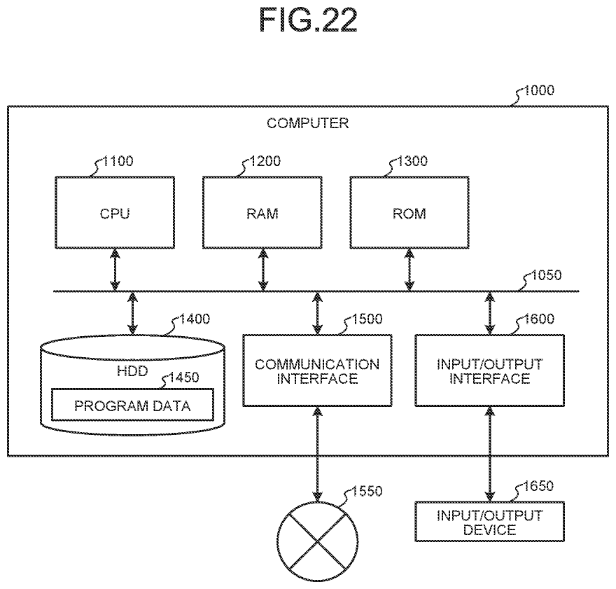

The information processing device such as the learning device 200 according to the embodiments explained above is realized by, for example, a computer 1000 having a configuration illustrated in FIG. 22. The learning device 200 according to an embodiment of the present disclosure is explained below as an example. FIG. 22 is a hardware configuration diagram illustrating an example of a computer that realizes a function of generating a learning model for teaching negative cases according to the embodiment of the present disclosure. The computer 1000 includes a CPU 1100, a RAM 1200, a ROM (Read Only Memory) 1300, a HDD (Hard Disk Drive) 1400, a communication interface 1500, and an input/output interface 1600. The units of the computer 1000 are connected by a bus 1050.

The CPU 1100 operates based on programs stored in the ROM 1300 or the HDD 1400 and controls the units. For example, the CPU 1100 develops, in the RAM 1200, the programs stored in the ROM 1300 or the HDD 1400 and executes processing corresponding to various programs.

The ROM 1300 stores a boot program such as a BIOS (Basic Input Output System) to be executed by the CPU 1100 at a start time of the computer 1000, a program depending on hardware of the computer 1000, and the like.

The HDD 1400 is a computer-readable recording medium that non-transiently records a program to be executed by the CPU 1100, data used by such a program, and the like. Specifically, the HDD 1400 is a recording medium that records a program for a medical arm control method according to the present disclosure, which is an example of program data 1450.

The communication interface 1500 is an interface for the computer 1000 to be connected to an external network 1550 (for example, the Internet). For example, the CPU 1100 receives data from other equipment and transmits data generated by the CPU 1100 to the other equipment via the communication interface 1500.

The input/output interface 1600 is an interface for connecting an input/output device 1650 and the computer 1000. For example, the CPU 1100 receives data from an input device such as a keyboard or a mouse via the input/output interface 1600. The CPU 1100 transmits data to an output device such as a display, a speaker, or a printer via the input/output interface 1600. The input/output interface 1600 may function as a media interface that reads a program or the like recorded in a predetermined computer-readable recording medium (a medium). The medium is, for example, an optical recording medium such as a DVD (Digital Versatile Disc) or a PD (Phase change rewritable Disk), a magneto-optical recording medium such as an MO (Magneto-Optical Disk), a tape medium, a magnetic recording medium, or a semiconductor memory.

For example, when the computer 1000 functions as the learning device 200 according to the embodiment of the present disclosure, the CPU 1100 of the computer 1000 executes a program for generating a learning model for teaching negative cases loaded on the RAM 1200 to thereby realize a function of generating the learning model for teaching negative cases. The HDD 1400 may store a program for generating a teacher model according to the embodiment of the present disclosure. Note that the CPU 1100 reads the program data 1450 from the HDD 1400 and executes the program data 1450. However, as another example, the CPU 1100 may acquire the information processing program from another device via the external network 1550.

The learning device 200 according to the present embodiment may be applied to a system including a plurality of devices premised on connection to a network (or communication between devices) such as cloud computing.

An example of the hardware configuration of the learning device 200 is explained above. The components explained above may be configured using general-purpose members or may be configured by hardware specialized for the functions of the components. Such a configuration can be changed as appropriate according to a technical level at each time to be implemented.

### 10. Supplement

Note that the embodiment of the present disclosure explained above can include, for example, an information processing method executed by the information processing device or the information processing system explained above, a program for causing the information processing device to function, and a non-transitory tangible medium in which the program is recorded. The program may be distributed via a communication line (including wireless communication) such as the Internet.

The steps in the information processing method in the embodiment of the present disclosure explained above may not always be processed according to the described order. For example, the steps may be processed with the order changed as appropriate. The steps may be partially processed in parallel or individually instead of being processed in time series. Further, the processing of the steps may not always be processed according to the described method and may be processed by, for example, another functional unit according to another method.

Among the kinds of processing explained in the above embodiments, all or a part of the processing explained as being automatically performed can be manually performed or all or a part of the processing explained as being manually performed can be automatically performed by a publicly-known method. Besides, the processing procedure, the specific names, and the information including the various data and parameters explained in the document and illustrated in the drawings can be optionally changed except when specifically noted otherwise. For example, the various kinds of information illustrated in the figures are not limited to the illustrated information.

The illustrated components of the devices are functionally conceptual and are not always required to be physically configured as illustrated in the figures. That is, specific forms of distribution and integration of the devices are not limited to the illustrated forms and all or a part thereof can be configured by being functionally or physically distributed and integrated in any unit according to various loads, usage situations, and the like.

The preferred embodiment of the present disclosure is explained in detail above with reference to the accompanying drawings. However, the technical scope of the present disclosure is not limited to such an example. It is evident that those having the ordinary knowledge in the technical field of the present disclosure can arrive at various alterations or corrections within the category of the technical idea described in claims. It is understood that these alterations and corrections naturally belong to the technical scope of the present disclosure.

The effects described in the present specification are only explanatory or illustrative and are not limiting. That is, the technique according to the present disclosure can achieve other effects obvious for those skilled in the art from the description of the present specification together with or instead of the effects described above.

Note that the present technique can also take the following configurations.

(1) An information processing device comprising a control unit that performs control of a medical arm to autonomously operate using a first learning model generated by machine learning a plurality of state information concerning an operation of the medical arm labeled as being an operation that should be avoided.
(2) The information processing device according to (1), further comprising a first machine learning unit that generates the first learning model.
(3) The information processing device according to (1) or (2), wherein the medical arm supports a medical observation device.
(4) The information processing device according to (3), wherein the medical observation device is an endoscope.
(5) The information processing device according to (1), wherein the medical arm supports a medical instrument.
(6) The information processing device according to any one of (1) to (5), wherein the plurality of state information includes at least any one of information among a position, a posture, speed, acceleration, and an image of the medical arm.
(7) The information processing device according to (6), wherein the plurality of state information includes information concerning different states of a same kind.
(8) The information processing device according to any one of (1) to (7), wherein the plurality of state information includes biological information of an operator.
(9) The information processing device according to (8), wherein the biological information includes at least any one of uttered voice, a motion, a line of sight, a heartbeat, a pulse, a blood pressure, a brain wave, respiration, sweating, myoelectric potential, skin temperature, and skin electrical resistance of the operator.
(10) The information processing device according to (2), wherein the first learning model estimates information concerning at least any one of a position, a posture, speed, acceleration of the medical arm, a feature value of an image, and an imaging condition.
(11) The information processing device according to (2), wherein the control unit causes the medical arm to autonomously operate to avoid a state estimated by the first learning model.
(12) The information processing device according to (11), further comprising an operation target determination unit that determines an operation target of the medical arm, wherein the control unit causes the medical arm to autonomously operate based on the operation target.
(13) The information processing device according to (11), further comprising
a state information acquisition unit that acquires a plurality of the state information; and
a first extraction unit that extracts, based on the first learning model, from the plurality of state information, a plurality of state information labeled as being an operation that may not be avoided.
   (14) The information processing device according to (13), further comprising a second machine learning unit that performs machine learning on the plurality of state information labeled as being the operation that may not be avoided and generates a second learning model.
   (15) The information processing device according to (14), wherein the control unit causes the medical arm to autonomously operate using the second learning model.
   (16) The information processing device according to (15), wherein the control unit performs weighting on the estimation of the first and second learning models.
   (17) The information processing device according to (15), wherein the control unit causes the medical arm to autonomously operate according to the first learning model and, subsequently, causes the medical arm to autonomously operate according to the second learning model.
   (18) The information processing device according to (2), further comprising:
a state information acquisition unit that acquires a plurality of the state information; and
a second extraction unit that extracts, from the plurality of state information, a plurality of state information labeled as being an operation that should be avoided.
   (19) The information processing device according to (18), wherein the second extraction unit extracts, based on any one of an image, uttered voice, and stop operation information included in the plurality of state information, from the plurality of state information, the plurality of state information labeled as being the operation that should be avoided.
   (20) The information processing device according to (2), further comprising an evaluation unit that evaluates an operation of the medical arm according to the first learning model.
   (21) A program for causing a computer to execute control of an autonomous operation of a medical arm using a first learning model generated by machine learning a plurality of state information concerning an operation of the medical arm labeled as an operation that should be avoided.
   (22) A learning model for causing a computer to function to perform control of a medical arm to autonomously operate to avoid a state output based on the learning model, the learning model comprising information concerning a feature value extracted by machine learning a plurality of state information concerning an operation of the medical arm labeled as an operation that should be avoided.
   (23) A method of generating a learning model for causing a computer to function to control a medical arm to autonomously operate to avoid a state output based on the learning model, the method comprising generating the learning model by machine learning a plurality of state information concerning an operation of the medical arm labeled as an operation that the medical arm should avoid.

### REFERENCE SIGNS LIST

10 MEDICAL OBSERVATION SYSTEM
100 ENDOSCOPIC ROBOT ARM SYSTEM
102 ARM UNIT
104 IMAGING UNIT
106 LIGHT SOURCE UNIT
200, 200a LEARNING DEVICE
212, 312, 412 INFORMATION ACQUISITION UNIT
214, 214a EXTRACTION UNIT
216, 216a MACHINE LEARNING UNIT
226, 326, 426 OUTPUT UNIT
230, 330, 430 STORAGE UNIT
300 CONTROL DEVICE
310 PROCESSING UNIT
314 IMAGE PROCESSING UNIT
316 TARGET STATE CALCULATION UNIT
318 FEATURE VALUE CALCULATION UNIT
320 LEARNING MODEL FOR TEACHING NEGATIVE CASES ACQUISITION UNIT
322 TEACHER MODEL ACQUISITION UNIT
324 INTEGRATION PROCESSING UNIT
400 EVALUATION DEVICE
414 EVALUATION CALCULATION UNIT
420 MODEL ACQUISITION UNIT
500 PRESENTATION DEVICE
600 SURGEON SIDE DEVICE
602 SENSOR
604 UI
700 SURGICAL VIDEO
702, 704 EVALUATION DISPLAY
800 MEDICAL INSTRUMENT
900 CURSOR
902 BUTTON

1. An information processing device comprising a control unit that performs control of a medical arm to autonomously operate using a first learning model generated by machine learning a plurality of state information concerning an operation of the medical arm labeled as being an operation that should be avoided.

2. The information processing device according to claim 1, further comprising a first machine learning unit that generates the first learning model.

3. The information processing device according to claim 1, wherein the medical arm supports a medical observation device.

4. The information processing device according to claim 3, wherein the medical observation device is an endoscope.

5. The information processing device according to claim 1, wherein the medical arm supports a medical instrument.

6. The information processing device according to claim 1, wherein the plurality of state information includes at least any one of information among a position, a posture, speed, acceleration, and an image of the medical arm.

7. The information processing device according to claim 6, wherein the plurality of state information includes information concerning different states of a same kind.

8. The information processing device according to claim 1, wherein the plurality of state information includes biological information of an operator.

9. The information processing device according to claim 8, wherein the biological information includes at least any one of uttered voice, a motion, a line of sight, a heartbeat, a pulse, a blood pressure, a brain wave, respiration, sweating, myoelectric potential, skin temperature, and skin electrical resistance of the operator.

10. The information processing device according to claim 2, wherein the first learning model estimates information concerning at least any one of a position, a posture, speed, acceleration of the medical arm, a feature value of an image, and an imaging condition.

11. The information processing device according to claim 2, wherein the control unit causes the medical arm to autonomously operate to avoid a state estimated by the first learning model.

12. The information processing device according to claim 11, further comprising an operation target determination unit that determines an operation target of the medical arm, wherein
the control unit causes the medical arm to autonomously operate based on the operation target.

13. The information processing device according to claim 11, further comprising
a state information acquisition unit that acquires a plurality of the state information; and
a first extraction unit that extracts, based on the first learning model, from the plurality of state information, a plurality of state information labeled as being an operation that may not be avoided.

14. The information processing device according to claim 13, further comprising a second machine learning unit that performs machine learning on the plurality of state information labeled as being the operation that may not be avoided and generates a second learning model.

15. The information processing device according to claim 14, wherein the control unit causes the medical arm to autonomously operate using the second learning model.

16. The information processing device according to claim 15, wherein the control unit performs weighting on the estimation of the first and second learning models.

17. The information processing device according to claim 15, wherein the control unit causes the medical arm to autonomously operate according to the first learning model and, subsequently, causes the medical arm to autonomously operate according to the second learning model.

18. The information processing device according to claim 2, further comprising:
a state information acquisition unit that acquires a plurality of the state information; and
a second extraction unit that extracts, from the plurality of state information, a plurality of state information labeled as being an operation that should be avoided.

19. The information processing device according to claim 18, wherein the second extraction unit extracts, based on any one of an image, uttered voice, and stop operation information included in the plurality of state information, from the plurality of state information, the plurality of state information labeled as being the operation that should be avoided.

20. The information processing device according to claim 2, further comprising an evaluation unit that evaluates an operation of the medical arm according to the first learning model.

21. A program for causing a computer to
execute control of an autonomous operation of a medical arm using a first learning model generated by machine learning a plurality of state information concerning an operation of the medical arm labeled as an operation that should be avoided.

22. A learning model for causing a computer to function to perform control of a medical arm to autonomously operate to avoid a state output based on the learning model, the learning model comprising
information concerning a feature value extracted by machine learning a plurality of state information concerning an operation of the medical arm labeled as an operation that should be avoided.

23. A method of generating a learning model for causing a computer to function to control a medical arm to autonomously operate to avoid a state output based on the learning model, the method comprising
generating the learning model by machine learning a plurality of state information concerning an operation of the medical arm labeled as an operation that the medical arm should avoid.
